SEQUENCE LISTING (20 sequences)

SEQ ID NO: 1 - DNA, 399 bp, Homo sapiens
gcgccgaccagctcatctacaaagaagactcaacttcaactggaacatctgctgcttgatctccaaatgattctcaacggtatcaacaattacaaaaacccaaaattgaccagaatgttgacatttaagttttacatgccgaaaaaggcaacagagctgaagcacttgcagtgtcttgaagaagagctgaaaccacttgaagaggttctgaacctggcacagagcaaaaattttcaccttaggccgcgcgacctcattagtaacattaatgtcattgtgcttgagctgaaaggttccgagacgactttcatgtgcgagtatgccgacgaaacggcaacaatagtcgaatttcttaaccgatggattacgttttgtcaaagtataataagtacgctcaca

SEQ ID NO: 2 - protein, 133 aa, Homo sapiens [sequence omitted]

SEQ ID NO: 3 - DNA, 399 bp, Artificial
  synthetic oligonucleotide
gcgccgaccagcagcagcaccaaaaaaacccagctgcagctggaacatctgctgctggatctgcagatgattctgaacggcattaacaactataaaaacccgaaactgaccgcgatgctgaccaaaaaattttatatgccgaaaaaagcgaccgaactgaaacatctgcagtgcctggaagaagaactgaaaccgctggaagaagtgctgaacctggcgcagagcaaaaactttcatctggacccgcgcgatgtggttagcaacattaacgtgtttgtgctggaactgaaaggcagcgaaaccacctttatgtgcgaatatgcggatgaaaccgcgaccattgtggaatttctgaaccgctggattaccttttgccagagcattattagcaccctgacc

SEQ ID NO: 4 - protein, 133 aa, Artificial
  synthetic peptide
APTSSSTKKTQLQLEHLLLDLQMILNGINNYKNPKLTAMLTKKFYMPKKATELKHLQCLEEELKPLEEVLNLAQSKNFHLDPRDVVSNINVFVLELKGSETTFMCEYADETATIVEFLNRWITFCQSIISTLT

SEQ ID NO: 5 - DNA, 399 bp, Artificial
  synthetic oligonucleotide
gcgccgaccagcagcagcaccaaaaaaacccagctgcagctggaacatctgctgctggatctgcagatgattctgaacggcattaacaactataaaaacccgaaactgaccgcgatgctgaccaaaaaattttatatgccgaaaaaagcgaccgaactgaaacatctgcagtgcctggaagaagaactgaaaccgctggaagaagtgctgaacctggcgcagagcaaaaactttcatttccgcccgcgcgatgtggttagcaacattaacgtgtttgtgctggaactgaaaggcagcgaaaccacctttatgtgcgaatatgcggatgaaaccgcgaccattgtggaatttctgaaccgctggattaccttttgccagagcattattagcaccctgacc

SEQ ID NO: 6 - protein, 133 aa, Artificial
  synthetic peptide
APTSSSTKKTQLQLEHLLLDLQMILNGINNYKNPKLTAMLTKKFYMPKKATELKHLQCLEEELKPLEEVLNLAQSKNFHFRPRDVVSNINVFVLELKGSETTFMCEYADETATIVEFLNRWITFCQSIISTLT

SEQ ID NO: 7 - DNA, 399 bp, Artificial
  synthetic oligonucleotide
gcgccgaccagcagcagcaccaaaaaaacccagctgcagctggaacatctgctgctggatctgcagatgattctgaacggcattaacaactataaaaacccgaaactgaccgcgatgctgaccaaaaaattttatatgccgaaaaaagcgaccgaactgaaacatctgcagtgcctggaagaagaactgaaaccgctggaagaagtgctgaacctggcgcagagcaaaaactttcatttcgacccgcgcgatctggttagcaacattaacgtgtttgtgctggaactgaaaggcagcgaaaccacctttatgtgcgaatatgcggatgaaaccgcgaccattgtggaatttctgaaccgctggattaccttttgccagagcattattagcaccctgacc

SEQ ID NO: 8 - protein, 133 aa, Artificial
  synthetic peptide
APTSSSTKKTQLQLEHLLLDLQMILNGINNYKNPKLTAMLTKKFYMPKKATELKHLQCLEEELKPLEEVLNLAQSKNFHFDPRDLVSNINVFVLELKGSETTFMCEYADETATIVEFLNRWITFCQSIISTLT

SEQ ID NO: 9 - DNA, 399 bp, Artificial
  synthetic oligonucleotide
gcgccgaccagcagcagcaccaaaaaaacccagctgcagctggaacatctgctgctggatctgcagatgattctgaacggcattaacaactataaaaacccgaaactgaccgcgatgctgaccaaaaaattttatatgccgaaaaaagcgaccgaactgaaacatctgcagtgcctggaagaagaactgaaaccgctggaagaagtgctgaacctggcgcagagcaaaaactttcatttcgacccgcgcgatgtgattagcaacattaacgtgtttgtgctggaactgaaaggcagcgaaaccacctttatgtgcgaatatgcggatgaaaccgcgaccattgtggaatttctgaaccgctggattaccttttgccagagcattattagcaccctgacc

SEQ ID NO: 10 - protein, 133 aa, Artificial
  synthetic peptide
APTSSSTKKTQLQLEHLLLDLQMILNGINNYKNPKLTAMLTKKFYMPKKATELKHLQCLEEELKPLEEVLNLAQSKNFHFDPRDVISNINVFVLELKGSETTFMCEYADETATIVEFLNRWITFCQSIISTLT

SEQ ID NO: 11 - DNA, 399 bp, Artificial
  synthetic oligonucleotide
gcgccgaccagcagcagcaccaaaaaaacccagctgcagctggaacatctgctgctggatctgcagatgattctgaacggcattaacaactataaaaacccgaaactgaccgcgatgctgaccaaaaaattttatatgccgaaaaaagcgaccgaactgaaacatctgcagtgcctggaagaagaactgaaaccgctggaagaagtgctgaacctggcgcagagcaaaaactttcatttcgacccgcgcgatgtggttagcaacattaacgtgattgtgctggaactgaaaggcagcgaaaccacctttatgtgcgaatatgcggatgaaaccgcgaccattgtggaatttctgaaccgctggattaccttttgccagagcattattagcaccctgacc

SEQ ID NO: 12 - protein, 133 aa, Artificial
  synthetic peptide
APTSSSTKKTQLQLEHLLLDLQMILNGINNYKNPKLTAMLTKKFYMPKKATELKHLQCLEEELKPLEEVLNLAQSKNFHFDPRDVVSNINVIVLELKGSETTFMCEYADETATIVEFLNRWITFCQSIISTLT

SEQ ID NO: 13 - DNA, 399 bp, Artificial
  synthetic oligonucleotide
gcgccgaccagcagcagcaccaaaaaaacccagctgcagctggaacatctgctgctggatctgcagatgattctgaacggcattaacaactataaaaacccgaaactgaccgcgatgctgaccttcaaattttatatgccgaaaaaagcgaccgaactgaaacatctgcagtgcctggaagaagaactgaaaccgctggaagaagtgctgaacctggcgcagagcaaaaactttcatttcgacccgcgcgatgtgattagcaacattaacgtgtttgtgctggaactgaaaggcagcgaaaccacctttatgtgcgaatatgcggatgaaaccgcgaccattgtggaatttctgaaccgctggattaccttttgccagagcattattagcaccctgacc

SEQ ID NO: 14 - protein, 133 aa, Artificial
  synthetic peptide
APTSSSTKKTQLQLEHLLLDLQMILNGINNYKNPKLTAMLTFKFYMPKKATELKHLQCLEEELKPLEEVLNLAQSKNFHFDPRDVISNINVFVLELKGSETTFMCEYADETATIVEFLNRWITFCQSIISTLT

SEQ ID NO: 15 - DNA, 399 bp, Artificial
  synthetic oligonucleotide
gcgccgaccagcagcagcaccaaaaaaacccagctgcagctggaacatctgctgctggatctgcagatgattctgaacggcattaacaactataaaaacccgaaactgacccgcatgctgaccgcgaaattttatatgccgaaaaaagcgaccgaactgaaacatctgcagtgcctggaagaagaactgaaaccgctggaagaagtgctgaacctggcgcagagcaaaaactttcattttgatccgcgcgatgtgattagcaacattaacgtgtttgtgctggaactgaaaggcagcgaaaccacctttatgtgcgaatatgcggatgaaaccgcgaccattgtggaatttctgaaccgctggattaccttttgccagagcattattagcaccctgacc

SEQ ID NO: 16 - protein, 133 aa, Artificial
  synthetic peptide
APTSSSTKKTQLQLEHLLLDLQMILNGINNYKNPKLTRMLTAKFYMPKKATELKHLQCLEEELKPLEEVLNLAQSKNFHFDPRDVISNINVFVLELKGSETTFMCEYADETATIVEFLNRWITFCQSIISTLT

SEQ ID NO: 17 - DNA, 399 bp, Artificial
  synthetic oligonucleotide
gcgccgaccagcagcagcaccaaaaaaacccagctgcagctggaacatctgctgctggatctgcagatgattctgaacggcattaacaactataaaaacccgaaactgaccgcgatgctgaccgcgaaattttatatgccgaaaaaagcgaccgaactgaaacatctgcagtgcctggaagaagaactgaaaccgctggaagaagtgctgaacctggcgcagagcaaaaactttcatttcgacccgcgcgatgtgattagcaacattaacgtgtttgtgctggaactgaaaggcagcgaaaccacctttatgtgcgaatatgcggatgaaaccgcgaccattgtggaatttctgaaccgctggattaccttttgccagagcattattagcaccctgacc

SEQ ID NO: 18 - protein, 133 aa, Artificial
  synthetic peptide
APTSSSTKKTQLQLEHLLLDLQMILNGINNYKNPKLTAMLTAKFYMPKKATELKHLQCLEEELKPLEEVLNLAQSKNFHFDPRDVISNINVFVLELKGSETTFMCEYADETATIVEFLNRWITFCQSIISTLT

SEQ ID NO: 19 - DNA, 720 bp, Artificial
  synthetic oligonucleotide
agcaacactaaagtcgacaagcgagtagaaccgaaatcatgcgacaaaacacatacgtgccctccctgcccagcaccacctgtcgcgggcccctctgttttcctgtttccacccaagccaaaggacacattgatgatttcccggactcctgaagtcacctgcgtggtagtagatgtatcacatgaagatccagaagtccagttcaactggtatgtggacggagtagaggtacataatgccaagaccaaaccacgggaagagcagttcaacagtactttccgggtagttagcgttttgactgtcgtacaccaagactggcttaatggaaaagaatacaagtgtaaggtaagcaacaagggcctgccggctccgatagagaaaaccattagcaagacaaagggccaaccacgcgaaccccaggtatataccctcccaccgtcccgcgaggagatgactaagaatcaagtttctctcacgtgcttggtaaagggcttctatccgagcgatatagccgtggagtgggagtctaatggtcagcccgaaaacaattacaaaactacgcctcctatgctggacagtgatgggagcttctttctttacagtaagcttaccgtggacaagtctcggtggcaacaaggaaatgtttttagttgttctgtaatgcatgaagcacttcataaccattacacccagaaaagtctgagcttgtccccgggaaaa

SEQ ID NO: 20 - protein, 240 aa, Artificial
  synthetic peptide
SNTKVDKRVEPKSCDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2023/0242607 A1
(43) Pub. Date: Aug. 3, 2023

(54) IL-2 VARIANTS

(71) Applicant: EUTILEX CO., LTD., Seoul (KR)

(72) Inventors: Byoung S. KWON, Seoul (KR); Seoung Hyun LEE, Seoul (KR); Sun Woo IM, Seoul (KR); Jin Kyung CHOI, Seoul (KR); Hyun Tae SON, Seoul (KR); Joong Won LEE, Seoul (KR); Hanna LEE, Seoul (KR); Sunhee HWANG, Seoul (KR)

(73) Assignee: EUTILEX CO., LTD., Seoul (KR)

(21) Appl. No.: 18/004,575

(22) PCT Filed: Jul. 7, 2021
(86) PCT No.: PCT/IB2021/056106

§ 371 Date: Jan. 6, 2023

Related U.S. Application Data
(60) Provisional application No. 63/050,068, filed on Jul. 9, 2020.

(51) Int. Cl. C07K 14/55; A61P 35/00

(52) U.S. Cl. CPC C07K 14/55; A61P 35/00; A61K 38/00; C07K 2319/30

(57) ABSTRACT

An interleukin (IL)-2 protein including a sequence selected from the group consisting of: SEQ ID NO: 4, SEQ ID NO: 6, SEQ ID NO: 8, SEQ ID NO: 10, SEQ ID NO: 12, SEQ ID NO: 14, SEQ ID NO: 16, and SEQ ID NO: 18. A pharmaceutical composition including the interleukin-2 protein and a pharmaceutically acceptable carrier.

### CROSS-REFERENCE TO RELATED APPLICATIONS

This application claims priority to U.S. Application Serial No. 63/050,068, filed on Jul. 9, 2020. The disclosure of the prior application is considered part of the disclosure of this application, and is incorporated in its entirety into this application.

### BACKGROUND

Cancer remains one of the leading causes of death in the world. Recent statistics report that 13% of the world population dies from cancer. According to estimates from the International Agency for Research on Cancer (IARC), in 2012 there were 14.1 million new cancer cases and 8.2 million cancer deaths worldwide. By 2030, the global burden is expected to grow to 21.7 million new cancer cases and 13 million cancer deaths due to population growth and aging and exposure to risk factors such as smoking, unhealthy diet and physical inactivity. Further, pain and medical expenses for cancer treatment cause reduced quality of life for both cancer patients and their families.

Interleukin-2 (IL-2) is a cytokine signaling molecule acting on the immune system to generate a cell-mediated immune response. Because of its essential role in the growth and differentiation of T cells, IL-2 has been a candidate in immunotherapeutic approaches for treating diseases such as cancers.

### SUMMARY

Provided herein are interleukin (IL)-2 proteins comprising: a sequence selected from the group consisting of: SEQ ID NO: 4, SEQ ID NO: 6, SEQ ID NO: 8, SEQ ID NO: 10, SEQ ID NO: 12, SEQ ID NO: 14, SEQ ID NO: 16, or SEQ ID NO: 18. In some embodiments, the interleukin-2 protein comprises SEQ ID NO: 4. In some embodiments, the interleukin-2 protein comprises SEQ ID NO: 6. In some embodiments, the interleukin-2 protein comprises SEQ ID NO: 8. In some embodiments, the interleukin-2 protein comprises SEQ ID NO: 10. In some embodiments, the interleukin-2 protein comprises SEQ ID NO: 12. In some embodiments, the interleukin-2 protein comprises SEQ ID NO: 14. In some embodiments, the interleukin-2 protein comprises SEQ ID NO: 16. In some embodiments, the interleukin-2 protein comprises SEQ ID NO: 18.

In some embodiments, any one of the interleukin-2 proteins described herein further comprise an immunoglobulin Fc region. In some embodiments, the immunoglobulin Fc region comprises SEQ ID NO: 20. In some embodiments, the immunoglobulin Fc region is linked to the interleukin-2 protein by a peptide bond. In some embodiments, the immunoglobulin Fc region is linked to the interleukin-2 protein by a peptide linker sequence. In some embodiments, the interleukin-2 protein is linked to the carboxy-terminus of the immunoglobulin Fc region.

Also provided herein are pharmaceutical compositions comprising any one of the proteins described herein and a pharmaceutically acceptable carrier.

Also provided herein are nucleic acids comprising: a sequence encoding an IL-2 protein, wherein the sequence is selected from the group consisting of: SEQ ID NO: 3, SEQ ID NO: 5, SEQ ID NO: 7, SEQ ID NO: 9, SEQ ID NO: 11, SEQ ID NO: 13, SEQ ID NO: 15, or SEQ ID NO: 17. In some embodiments, any one of the nucleic acids described herein further comprise a sequence encoding an immunoglobulin Fc region. In some embodiments, the sequence encoding the immunoglobulin Fc region comprises SEQ ID NO: 19. In some embodiments, the sequence encoding the IL-2 protein comprises SEQ ID NO: 3. In some embodiments, the sequence encoding the IL-2 protein comprises SEQ ID NO: 5. In some embodiments, the sequence encoding the IL-2 protein comprises SEQ ID NO: 7. In some embodiments, the sequence encoding the IL-2 protein comprises SEQ ID NO: 9. In some embodiments, the sequence encoding the IL-2 protein comprises SEQ ID NO: 11. In some embodiments, the sequence encoding the IL-2 protein comprises SEQ ID NO: 13. In some embodiments, the sequence encoding the IL-2 protein comprises SEQ ID NO: 15. In some embodiments, the sequence encoding the IL-2 protein comprises SEQ ID NO: 17.

Also provided herein are vectors comprising any one of the nucleic acids described herein. In some embodiments, any one of the vectors described herein further comprise a promoter operationally linked to the nucleic acid. In some embodiments, the promoter is a constitutive promoter. In some embodiments, the promoter is an inducible promoter. In some embodiments, the vector is a viral vector. In some embodiments, the viral vector is a lentiviral vector.

Also provided herein are pharmaceutical compositions comprising any one of the nucleic acids described herein or any one of the vectors described herein.

Also provided herein are cells comprising any one of the nucleic acids described herein or any one of the vectors described herein.

Also provided herein are pharmaceutical compositions comprising any one of the cells described herein and a pharmaceutically acceptable carrier.

Also provided herein are methods of producing a IL-2 protein, comprising: (a) culturing any one of the cells described herein in a culture medium under conditions sufficient to express the IL-2 protein; and (b) recovering the IL-2 protein from the cell and/or the culture medium.

Also provided herein are methods of treating a cancer in a subject, the method comprising administering to the subject a therapeutically effective amount of any one of the pharmaceutical compositions described herein. In some embodiments, the cancer is carcinoma, lymphoma (e.g., Hodgkin’s and non-Hodgkin’s lymphomas), blastoma, sarcoma, leukemia, squamous cell carcinoma, small cell lung cancer, non-small cell lung cancer, lung adenocarcinoma, squamous cell carcinoma of the lung, peritoneal cancer, hepatocellular carcinoma, gastric cancer, pancreatic cancer, glioma, cervical cancer, ovarian cancer, liver cancer, bladder cancer, breast cancer, colon cancer, colorectal cancer, endometrial or uterine carcinoma, salivary carcinoma, kidney cancer, prostate cancer, vulvar cancer, thyroid cancer, liver carcinoma, other lymphoproliferative disorders, or various types of head and neck cancer. In some embodiments, the cancer is a solid tumor. In some embodiments, the cancer is colorectal cancer.

In some embodiments, the subject has previously been administered one or more additional anticancer therapies selected from the group consisting of: ionizing radiation, a chemotherapeutic agent, a therapeutic antibody, and a checkpoint inhibitor. In some embodiments, the subject has been identified or diagnosed as having the cancer.

Also provided herein are methods of increasing memory CD8+ T cells in a subject, the method comprising administering to the subject a therapeutically effective amount of any one of the pharmaceutical compositions described herein.

Also provided herein are methods of increasing CD8+ T cells in a subject, the method comprising administering to the subject a therapeutically effective amount of any one of the pharmaceutical compositions described herein.

Also provided herein are methods of decreasing Treg cells in a solid tumor in a subject, the method comprising administering to the subject a therapeutically effective amount of any one of the pharmaceutical compositions described herein.

Also provided herein are methods of decreasing a rate of growth of a solid tumor in a subject, the method comprising administering to the subject a therapeutically effective amount of any one of the pharmaceutical compositions described herein.

Also provided herein are methods of decreasing volume of a solid tumor in a subject, the method comprising administering to the subject a therapeutically effective amount of any one of the pharmaceutical compositions described herein.

### BRIEF DESCRIPTION OF DRAWINGS

FIG. 1 shows the structure of a recombinant expression vector for an IL-2 fusion protein.

FIG. 2 shows SDS-PAGE results with IL-2 Fc fusion proteins, IL2_V1, IL2_V2, IL2_V3, IL2_V4, and IL2_V5.

FIG. 3A shows a size exclusion chromatography graph with gel filtration standard.

FIG. 3B shows a size exclusion chromatography graph with supMD.

FIG. 3C shows a size exclusion chromatography graph with IL2_V1.

FIG. 3D shows a size exclusion chromatography graph with IL2_V2.

FIG. 3E shows a size exclusion chromatography graph with IL2_V3.

FIG. 3F shows a size exclusion chromatography graph with IL2_V4.

FIG. 3G shows a size exclusion chromatography graph with IL2_V5.

FIG. 4 shows SDS-PAGE results with additional IL-2 Fc fusion proteins, IL2_V4, IL2_V4 R38A, IL2_V4_F42A, and IL2_V4_R38A_F42A.

FIG. 5A shows a size exclusion chromatography graph with gel filtration standard.

FIG. 5B shows a size exclusion chromatography graph with IL2_V4_R38A.

FIG. 5C shows a size exclusion chromatography graph with IL2_V4_F42A.

FIG. 5D shows a size exclusion chromatography graph with IL2_V4_R38A_F42A.

FIG. 6A is a graph showing individual tumor growth with IL2 WT.

FIG. 6B is a graph showing individual tumor growth with IL2 V4.

FIG. 6C is a graph showing individual tumor growth with IL2 V4_R38A.

FIG. 6D is a graph showing individual tumor growth with IL2 V4_F42A.

FIG. 6E is a graph showing individual tumor growth with IL2 V4_R38A_F42A.

FIG. 6F is a graph showing a tumor growth curve with IL2 WT, IL2 V4, IL2 V4_R38A, IL2 V4_F42A, and IL2 V4_R38A_F42A.

FIG. 6G is a bar graph showing tumor growth with IL2 WT, IL2 V4, IL2 V4_R38A, IL2 V4_F42A, and IL2 V4_R38A_F42A at the end point of the in vivo experiment.

FIG. 7A shows results from FACS analysis with IL2 WT, IL2 V4, IL2 V4_R38A, IL2 V4_F42A, and IL2 V4_R38A_F42A showing expression of CD3 and CD45.

FIG. 7B is a graph showing the percent of mCD3+ T cells with IL2 WT, IL2 V4, IL2 V4_R38A, IL2 V4_F42A, and IL2 V4_R38A_F42A.

FIG. 8A shows results from FACS analysis showing expression of CD4 and CD8 in the blood of mice treated with IL2 WT, IL2 V4, IL2 V4_R38A, IL2 V4_F42A, and IL2 V4_R38A_F42A.

FIG. 8B is a graph showing the percent of mCD8+ T cells in the blood of mice treated with IL2 WT, IL2 V4, IL2 V4 R38A, IL2 V4 F42A, and IL2 V4_R38A_F42A.

FIG. 9A shows results from FACS analysis showing expression of CD62L and CD44 in the blood of mice treated with IL2 WT, IL2 V4, IL2 V4_R38A, IL2 V4_F42A, and IL2 V4_R38A_F42A.

FIG. 9B is a graph showing the percent of mCD8+ central memory T cells in the blood of mice treated with IL2 WT, IL2 V4, IL2 V4_R38A, IL2 V4_F42A, and IL2 V4_R38A_F42A.

FIG. 9C is a graph showing the percent of mCD8+ effector T cells in the blood of mice treated with IL2 WT, IL2 V4, IL2 V4 R38A, IL2 V4 F42A, and IL2 V4_R38A_F42A.

FIG. 10A shows results from FACS analysis showing expression of CD4 and CD45 in the blood of mice treated with IL2 WT, IL2 V4, IL2 V4_R38A, IL2 V4_F42A, and IL2_V4_R38A_F42A.

FIG. 10B is a graph showing the percent of mCD4+ T cells in the blood of mice treated with IL2 WT, IL2 V4, IL2 V4 R38A, IL2 V4 F42A, and IL2 V4_R38A_F42A.

FIG. 11A shows results from FACS analysis showing expression of CD4 and Foxp3 in the blood of mice treated with IL2 WT, IL2 V4, IL2 V4_R38A, IL2 V4_F42A, and IL2_V4_R38A_F42A.

FIG. 11B is a graph showing the percent of Treg cells in the blood of mice treated with IL2 WT, IL2 V4, IL2 V4 R38A, IL2 V4 F42A, and IL2 V4_R38A_F42A.

FIG. 12A shows results from FACS analysis showing expression of CD3 and CD45 in tumor infiltrating lymphocytes of mice treated with IL2 WT, IL2 V4, IL2 V4_R38A, IL2 V4_F42A, and IL2 V4_R38A_F42A.

FIG. 12B is a graph showing the percent of mCD3+ T cells in tumor infiltrating lymphocytes of mice treated with IL2 WT, IL2 V4, IL2 V4_R38A, IL2 V4_F42A, and IL2_V4_R38A_F42A.

FIG. 13A shows results from FACS analysis showing expression of CD4 and CD8 in tumor infiltrating lymphocytes of mice treated with IL2 WT, IL2 V4, IL2 V4_R38A, IL2 V4_F42A, and IL2 V4_R38A_F42A.

FIG. 13B is a graph showing the percent of mCD8+ T cells in tumor infiltrating lymphocytes of mice treated with IL2 WT, IL2 V4, IL2 V4_R38A, IL2_V4_F42A, and IL2 V4_R38A_F42A.

FIG. 14A shows results from FACS analysis showing expression of CD62L and CD44 in tumor infiltrating lymphocytes of mice treated with IL2 WT, IL2 V4, IL2 V4_R38A, IL2 V4_F42A, and IL2 V4_R38A_F42A.

FIG. 14B is a graph showing the percent of mCD8+ central memory T cells in tumor infiltrating lymphocytes of mice treated with IL2 WT, IL2 V4, IL2 V4_R38A, IL2 V4_F42A, and IL2 V4_R38A_F42A.

FIG. 14C is a graph showing the percent of mCD8+ effector T cells in tumor infiltrating lymphocytes of mice treated with IL2 WT, IL2 V4, IL2 V4_R38A, IL2 V4_F42A, and IL2 V4_R38A_F42A.

FIG. 15A shows results from FACS analysis showing expression of CD4 and CD45 in tumor infiltrating lymphocytes of mice treated with IL2 WT, IL2 V4, IL2 V4_R38A, IL2 V4_F42A, and IL2 V4_R38A_F42A.

FIG. 15B is a graph showing the percent of mCD4+ T cells in tumor infiltrating lymphocytes of mice treated with IL2 WT, IL2 V4, IL2 V4_R38A, IL2 V4_F42A, and IL2 V4_R38A_F42A.

FIG. 16A shows results from FACS analysis showing expression of CD4 and Foxp3 in tumor infiltrating lymphocytes of mice treated with IL2 WT, IL2 V4, IL2 V4_R38A, IL2 V4_F42A, and IL2 V4_R38A_F42A.

FIG. 16B is a graph showing the percent of Treg cells in tumor infiltrating lymphocytes of mice treated with IL2 WT, IL2 V4, IL2 V4_R38A, IL2 V4_F42A, and IL2 V4_R38A_F42A.

FIG. 17A is a set of graphs showing the percent of mCD3+ T cells, mCD8+ T cells, mCD4+ T cells, mCD8+ central memory T cells, mCD8+ effector T cells, and Treg cells in the blood of mice treated with IL2 WT, IL2 V4, IL2 V4_R38A, IL2 V4_F42A, and IL2 V4_R38A_F42A.

FIG. 17B is a set of graphs showing the percent of mCD3+ T cells, mCD8+ T cells, mCD4+ T cells, mCD8+ central memory T cells, mCD8+ effector T cells, and Treg cells in tumor infiltrating lymphocytes of mice treated with IL2 WT, IL2 V4, IL2 V4_R38A, IL2 V4_F42A, and IL2 V4_R38A_F42A.

FIG. 18A show an exemplary image of lung tissue from C57BL/6 mice treated with IL2 WT, IL2 V4, IL2 V4_R38A, IL2 V4 F42A, and IL2 V4_R38A_F42A.

FIG. 18B is a graph showing the weight of lung tissue from C57BL/6 mice treated with IL2 WT, IL2 V4, IL2 V4 R38A, IL2 V4 F42A, and IL2 V4 R38A F42A.

FIG. 19A is a bar graph showing levels of gamma-glutamyl transpeptidase (GTP) in the blood serum of mice treated with IL2 WT, IL2 V4, IL2 V4_R38A, IL2 V4_F42A, and IL2 V4_R38A_F42A.

FIG. 19B is a bar graph showing levels of glutamic-oxaloacetic transaminase (GOT) in the blood serum of mice treated with IL2 WT, IL2 V4, IL2 V4_R38A, IL2 V4_F42A, and IL2 V4_R38A_F42A.

FIG. 19C is a bar graph showing levels of blood urea nitrogen (BUN) in the blood serum of mice treated with IL2 WT, IL2 V4, IL2 V4_R38A, IL2 V4_F42A, and IL2_V4_R38A_F42A.

FIG. 19D is a bar graph showing levels of total bilirubin (T-BIL) in the blood serum of mice treated with IL2 WT, IL2 V4, IL2 V4_R38A, IL2 V4_F42A, and IL2 V4_R38A_F42A.

### DETAILED DESCRIPTION

This disclosure describes variants of IL-2 protein, wherein the IL-2 variants include mutations in the human IL-2 protein.

### Definitions

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the strength a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies, polyclonal antibodies, and fragments thereof. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE, or IgM antibodies; bi- or multi- specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies;, Adnectins®; Affilins®; Trans-bodies®; Affibodies®; TrimerX®; MicroProteins; Fynomers®, Centyrins®; and KALBITOR®s. In some embodiments, an antibody agent may lack a covalent modification (e.g., attachment of a glycan) that it would have if produced naturally. In some embodiments, an antibody agent may contain a covalent modification (e.g., attachment of a glycan, a payload [e.g., a detectable moiety, a therapeutic moiety, a catalytic moiety, etc.], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent is or comprises at least a portion of a chimeric antigen receptor (CAR).

Antigen: The term “antigen”, as used herein, refers to an agent that binds to an antibody agent. In some embodiments, an antigen binds to an antibody agent and may or may not induce a particular physiological response in an organism. In general, an antigen may be or include any chemical entity such as, for example, a small molecule, a nucleic acid, a polypeptide, a carbohydrate, a lipid, a polymer (including biologic polymers [e.g., nucleic acid and/or amino acid polymers] and polymers other than biologic polymers [e.g., other than a nucleic acid or amino acid polymer]) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some certain embodiments, an antigen is present in a cellular context (e.g., an antigen is expressed on the surface of a cell or expressed in a cell). In some embodiments, an antigen is a recombinant antigen.

Antigen binding domain: As used herein, refers to an antibody agent or portion thereof that specifically binds to a target moiety or entity. Typically, the interaction between an antigen binding domain and its target is non-covalent. In some embodiments, a target moiety or entity can be of any chemical class including, for example, a carbohydrate, a lipid, a nucleic acid, a metal, a polypeptide, or a small molecule. In some embodiments, an antigen binding domain may be or comprise a polypeptide (or complex thereof). In some embodiments, an antigen binding domain is part of a fusion polypeptide. In some embodiments, an antigen binding domain is part of a chimeric antigen receptor (CAR).

Associated with: Two events or entities are “associated” with one another, as that term is used herein, if the presence, level, and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts―including where interacting entities or moieties are studied in isolation or in the context of more complex systems (e.g., while covalently or otherwise associated with a carrier entity and/or in a biological system or cell).

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin’s and non-Hodgkin’s), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Chemotherapeutic Agent: The term “chemotherapeutic agent”, has used herein has its art-understood meaning referring to one or more pro-apoptotic, cytostatic and/or cytotoxic agents, for example specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some particular embodiments, a chemotherapeutic agent may be or comprise one or more of Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g. DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, and combinations thereof. In some embodiments, a chemotherapeutic agent may be utilized in the context of an antibody-drug conjugate. In some embodiments, a chemotherapeutic agent is one found in an antibody-drug conjugate selected from the group consisting of: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine.

Engineered: In general, the term “engineered” refers to the aspect of having been manipulated by the hand of man. For example, a polypeptide is considered to be “engineered” when the polypeptide sequence manipulated by the hand of man. For example, in some embodiments of the present invention, an engineered polypeptide comprises a sequence that includes one or more amino acid mutations, deletions and/or insertions that have been introduced by the hand of man into a reference polypeptide sequence. In some embodiments, an engineered polypeptide includes a polypeptide that has been fused (i.e., covalently linked) to one or more additional polypeptides by the hand of man, to form a fusion polypeptide that would not naturally occur in vivo. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, derivatives and/or progeny of an engineered polypeptide or cell are typically still referred to as “engineered” even though the actual manipulation was performed on a prior entity.

Pharmaceutical composition: As used herein, the term “pharmaceutical composition” refers to a composition in which an active agent is formulated together with one or more pharmaceutically acceptable carriers. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

Polypeptide: The term “polypeptide”, as used herein, generally has its artrecognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibody agents, antibody fragments, biologically active portions thereof, and/or characteristic portions thereof.

Recombinant, as used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically Effective Amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, term “therapeutically effective amount”, refers to an amount which, when administered to an individual in need thereof in the context of inventive therapy, will block, stabilize, attenuate, or reverse a cancer-supportive process occurring in said individual, or will enhance or increase a cancer-suppressive process in said individual. In the context of cancer treatment, a “therapeutically effective amount” is an amount which, when administered to an individual diagnosed with a cancer, will prevent, stabilize, inhibit, or reduce the further development of cancer in the individual. A particularly preferred “therapeutically effective amount” of a composition described herein reverses (in a therapeutic treatment) the development of a malignancy such as a pancreatic carcinoma or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor such as a pancreatic adenocarcinoma (e.g., over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this invention would be indicated by finding a decrease in any of the pro-angiogenic markers discussed above, an increase in anti-angiogenic markers described herein, the normalization (i.e., alteration toward a state found in normal individuals not suffering from cancer) of metabolic pathways, intercellular signaling pathways, or intracellular signaling pathways that exhibit abnormal activity in individuals diagnosed with cancer. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence. In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid.

Vector, as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Standard techniques may be used for recombinant DNA, oligonucleotide synthesis, and tissue culture and transformation (e.g., electroporation, lipofection). Enzymatic reactions and purification techniques may be performed according to manufacturer’s specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual 2^{nd} ed., Cold Spring Harbor Laboratory Press, Cold Spring Harbor, N.Y. (1989)), which is incorporated herein by reference for any purpose.

### Interleukin-2 (IL-2)

Interleukin-2 (IL-2) is a cytokine signaling molecule in the immune system. IL-2 regulates the activities of white blood cells (e.g., leukocytes, lymphocytes), and mediates its effects by binding to IL-2 receptors (e.g., IL2-RA, IL2-RB) which are expressed by lymphocytes. Further, during an immune response, activated antigen-specific CD4⁺ and CD8⁺ T cells are the major sources of IL-2, which is then consumed by CD25⁺ effector T cells and T_{Reg} cells. Also, IL-2 has been shown to exert stimulatory and regulatory functions by binding to various IL-2 receptors, including monomeric, dimeric, and trimeric IL-2 receptors. Some T cells (e.g., T_{Reg} cells) express high-affinity heterotrimeric receptors composed of IL2-RA (CD25), IL2-RB (CD122), and the common cytokine receptor gamma chain (CD132) subunits. In contrast, naive CD8 T cells, CD4/CD8 memory T cells, and NK cells express a lower-affinity dimeric receptor, which lacks the IL2-RA subunit.

IL-2 has roles in key functions of the immune system, tolerance and immunity, primarily via its direct effects on T cells. IL-2 also promotes the differentiation of T cells into effector T cells and memory T cells when the initial T cell is also stimulated by an antigen. In some embodiments, IL-2 is produced by activated CD4⁺ T cells in secondary lymphoid organs. The secreted IL-2 is then consumed at the same site by regulatory T (T_{Reg}) cells that express the IL-2 receptor subunit CD25 (e.g., IL-2Rα). In some embodiments, during an immune response, activated antigen-specific CD4⁺ and CD8⁺ T cells produce large amounts of IL-2, which is then consumed by CD25⁺ effector T cells and T_{Reg} cells.

IL-2 has been shown to be produced by activated T cells in secondary lymphoid organs, where it is consumed by these cells and other CD25⁺ cells, including T_{Reg} cells. In some embodiments, the ability of IL-2 to activate both T_{Reg} cells and cytotoxic lymphocytes can be circumvented by using low-dose IL-2 immunotherapy to increase only T_{Reg} cell numbers in autoimmunity, chronic inflammatory conditions and graft rejection. In some embodiments, high-dose IL-2 administration can serve to expand cytotoxic lymphocyte populations for the treatment of metastatic cancer. An alternative approach for selective IL-2 immunotherapy would be to use improved IL-2 formulations, such as IL-2 bound to particular IL-2-specific monoclonal antibodies, or IL-2 muteins with increased affinity for CD25 or CD122 (e.g., IL-2Rβ. In some embodiments, specific mutations to IL-2 are introduced to produce IL-2 variants, wherein the specific mutations induce preferential binding to either IL2-RA or IL2-RB, thus stimulating specific immune cell subsets.

### Proteins

Provided herein are proteins including a sequence selected from the group consisting of: SEQ ID NO: 4, SEQ ID NO: 6, SEQ ID NO: 8, SEQ ID NO: 10, SEQ ID NO: 12, SEQ ID NO: 14, SEQ ID NO: 16, or SEQ ID NO: 18. In some embodiments, the protein includes SEQ ID NO: 4. In some embodiments, the protein includes SEQ ID NO: 6. In some embodiments, the protein includes SEQ ID NO: 8. In some embodiments, the protein includes SEQ ID NO: 10. In some embodiments, the protein includes SEQ ID NO: 12. In some embodiments, the protein includes SEQ ID NO: 14. In some embodiments, the protein includes SEQ ID NO: 16. In some embodiments, the protein includes SEQ ID NO: 18. In some embodiments, the protein further includes an immunoglobulin Fc region. In some embodiments, the immunoglobulin Fc region includes SEQ ID NO: 20. In some embodiments, the immunoglobulin Fc region is linked to the protein by a peptide bond. In some embodiments, the immunoglobulin Fc region is linked to the protein by a peptide linker sequence. In some embodiments, the protein is linked to the carboxy-terminus of the immunoglobulin Fc region. In some embodiments, the protein further includes one or more domains (e.g., one or more functional domains, e.g., cytokines).

In some embodiments, the protein is an IL-2 protein. In some embodiments, an IL-2 protein is a human interleukin-2 (IL-2) protein. In some embodiments, an IL-2 protein is a soluble IL-2 protein. In some embodiments, an IL-2 protein is a protein binder. In some embodiments, an IL-2 protein can be used in combination with another antibody agent. In some embodiments, an IL-2 protein can bind to an antibody agent. In some embodiments, an IL-2 protein is used in an IL-2 fusion protein. In some embodiments, an IL-2 fusion protein can include an IL-2 protein and an immunoglobulin Fc region. In some embodiments, the fusion protein includes a linker that fuses the Fc domain and specific peptide or protein together. In some embodiments, the immunoglobulin Fc region is linked to the specific peptide or protein by a peptide bond. In some embodiments, the immunoglobulin Fc Region is linked to the specific peptide or protein by a peptide linker sequence. In some embodiments, the specific peptide or protein is linked to the carboxy-terminus of the immunoglobulin Fc region. In some embodiments, an IL-2 protein includes SEQ ID NO: 2. In some embodiments, an IL-2 protein includes a sequence selected from the group consisting of: SEQ ID NO: 4, SEQ ID NO: 6, SEQ ID NO: 8, SEQ ID NO: 10, SEQ ID NO: 12, SEQ ID NO: 14, SEQ ID NO: 16, or SEQ ID NO: 18.

Provided herein are interleukin (IL)-2 proteins including a sequence selected from the group consisting of: SEQ ID NO: 4, SEQ ID NO: 6, SEQ ID NO: 8, SEQ ID NO: 10, SEQ ID NO: 12, SEQ ID NO: 14, SEQ ID NO: 16, or SEQ ID NO: 18. In some embodiments, the interleukin-2 protein includes SEQ ID NO: 4. In some embodiments, the interleukin-2 protein includes SEQ ID NO: 6. In some embodiments, the interleukin-2 protein includes SEQ ID NO: 8. In some embodiments, the interleukin-2 protein includes SEQ ID NO: 10. In some embodiments, the interleukin-2 protein includes SEQ ID NO: 12. In some embodiments, the interleukin-2 protein includes SEQ ID NO: 14. In some embodiments, the interleukin-2 protein includes SEQ ID NO: 16. In some embodiments, the interleukin-2 protein includes SEQ ID NO: 18. In some embodiments, the interleukin-2 protein further includes an immunoglobulin Fc region. In some embodiments, the immunoglobulin Fc region includes SEQ ID NO: 20. In some embodiments, the immunoglobulin Fc region is linked to the human interleukin-2 protein by a peptide bond. In some embodiments, the immunoglobulin Fc region is linked to the human interleukin-2 protein by a peptide linker sequence. In some embodiments, the interleukin-2 protein is linked to the carboxy-terminus of the immunoglobulin Fc region.

### Nucleic Acid

As used herein, “nucleic acid” is used to include any compound and/or substance that comprise polynucleotides. Exemplary nucleic acids or polynucleotides can include, but are not limited to, ribonucleic acids (RNAs) and/or deoxyribonucleic acids (DNAs).

Provided herein are nucleic acids including a sequence encoding an IL-2 protein, wherein the sequence is selected from the group consisting of: SEQ ID NO: 3, SEQ ID NO: 5, SEQ ID NO: 7, SEQ ID NO: 9, SEQ ID NO: 11, SEQ ID NO: 13, SEQ ID NO: 15, or SEQ ID NO: 17. In some embodiments, the nucleic acid further includes a sequence encoding an immunoglobulin Fc region. In some embodiments, the sequence encoding the immunoglobulin Fc region comprises SEQ ID NO: 19. In some embodiments, the sequence encoding the IL-2 protein comprises SEQ ID NO: 3. In some embodiments, the sequence encoding the IL-2 protein comprises SEQ ID NO: 5. In some embodiments, the sequence encoding the IL-2 protein comprises SEQ ID NO: 7. In some embodiments, the sequence encoding the IL-2 protein comprises SEQ ID NO: 9. In some embodiments, the sequence encoding the IL-2 protein comprises SEQ ID NO: 11. In some embodiments, the sequence encoding the IL-2 protein comprises SEQ ID NO: 13. In some embodiments, the sequence encoding the IL-2 protein comprises SEQ ID NO: 15. In some embodiments, the sequence encoding the IL-2 protein comprises SEQ ID NO: 17

SEQ ID NO: 1 - wild-type human IL-2 GCGCCGACCAGCTC
ATCTACAAAGAAGACTCAACTTCAACTGGAACATCTGCTGCTTGATCTCC
AAATGATTCTCAACGGTATCAACAATTACAAAAACCCAAAATTGACCAGA
ATGTTGACATTTAAGTTTTACATGCCGAAAAAGGCAACAGAGCTGAAGCA
CTTGCAGTGTCTTGAAGAAGAGCTGAAACCACTTGAAGAGGTTCTGAACC
TGGCACAGAGCAAAAATTTTCACCTTAGGCCGCGCGACCTCATTAGTAAC
ATTAATGTCATTGTGCTTGAGCTGAAAGGTTCCGAGACGACTTTCATGTG
CGAGTATGCCGACGAAACGGCAACAATAGTCGAATTTCTTAACCGATGGA
TTACGTTTTGTCAAAGTATAATAAGTACGCTCACA

SEQ ID NO: 2 - wild-type IL-2 (amino acid) APTSSST
KKTQLQLEHLLLDLQMILNGINNYKNPKLTRMLTFKFYMPKKATELKHLQ
CLEEELKPLEEVLNLAQSKNFHLRPRDLISNINVIVLELKGSETTFMCEY
ADETATIVEFLNRWITFCQSIISTLT

SEQ ID NO: 3 - IL2_V1GCGCCGACCAGCAGCAGCACCAAAAAAAC
CCAGCTGCAGCTGGAACATCTGCTGCTGGATCTGCAGATGATTCTGAACG
GCATTAACAACTATAAAAACCCGAAACTGACCGCGATGCTGACCAAAAAA
TTTTATATGCCGAAAAAAGCGACCGAACTGAAACATCTGCAGTGCCTGGA
AGAAGAACTGAAACCGCTGGAAGAAGTGCTGAACCTGGCGCAGAGCAAAA
ACTTTCATctgGACCCGCGCGATGTGGTTAGCAACATTAACGTGTTTGTG
CTGGAACTGAAAGGCAGCGAAACCACCTTTATGTGCGAATATGCGGATGA
AACCGCGACCATTGTGGAATTTCTGAACCGCTGGATTACCTTTTGCCAGA
GCATTATTAGCACCCTGACC

SEQ ID NO: 4 - IL2_V1 (amino acid) APTSSSTKKTQLQLE
HLLLDLQMILNGINNYKNPKLTAMLTKKFYMPKKATELKHLQCLEEELKP
LEEVLNLAQSKNFHLDPRDWSNINVFVLELKGSETTFMCEYADETATIVE
FLNRWITFCQSIISTLT

SEQ ID NO: 5 - IL2_V2 GCGCCGACCAGCAGCAGCACCAAAAAAA
CCCAGCTGCAGCTGGAACATCTGCTGCTGGATCTGCAGATGATTCTGAAC
GGCATTAACAACTATAAAAACCCGAAACTGACCGCGATGCTGACCAAAAA
ATTTTATATGCCGAAAAAAGCGACCGAACTGAAACATCTGCAGTGCCTGG
AAGAAGAACTGAAACCGCTGGAAGAAGTGCTGAACCTGGCGCAGAGCAAA
AACTTTCATTTCcgcCCGCGCGATGTGGTTAGCAACATTAACGTGTTTGT
GCTGGAACTGAAAGGCAGCGAAACCACCTTTATGTGCGAATATGCGGATG
AAACCGCGACCATTGTGGAATTTCTGAACCGCTGGATTACCTTTTGCCAG
AGCATTATTAGCACCCTGACC

SEQ ID NO: 6 - IL2_V2 (amino acid) APTSSSTKKTQLQLE
HLLLDLQMILNGINNYKNPKLTAMLTKKFYMPKKATELKHLQCLEEELKP
LEEVLNLAQSKNFHFRPRDWSNINVFVLELKGSETTFMCEYADETATIVE
FLNRWITFCQSIISTLT

SEQ ID NO: 7 - IL2_V3 GCGCCGACCAGCAGCAGCACCAAAAAAA
CCCAGCTGCAGCTGGAACATCTGCTGCTGGATCTGCAGATGATTCTGAAC
GGCATTAACAACTATAAAAACCCGAAACTGACCGCGATGCTGACCAAAAA
ATTTTATATGCCGAAAAAAGCGACCGAACTGAAACATCTGCAGTGCCTGG
AAGAAGAACTGAAACCGCTGGAAGAAGTGCTGAACCTGGCGCAGAGCAAA
AACTTTCATTTCGACCCGCGCGATctgGTTAGCAACATTAACGTGTTTGT
GCTGGAACTGAAAGGCAGCGAAACCACCTTTATGTGCGAATATGCGGATG
AAACCGCGACCATTGTGGAATTTCTGAACCGCTGGATTACCTTTTGCCAG
AGCATTATTAGCACCCTGACC

SEQ ID NO: 8 - IL2_V3 (amino acid) APTSSSTKKTQLQLE
HLLLDLQMILNGINNYKNPKLTAMLTKKFYMPKKATELKHLQCLEEELKP
LEEVLNLAQSKNFHFDPRDLVSNINVFVLELKGSETTFMCEYADETATIV
EFLNRWITFCQSIISTLT

SEQ ID NO: 9 - IL2_V4 GCGCCGACCAGCAGCAGCACCAAAAAAA
CCCAGCTGCAGCTGGAACATCTGCTGCTGGATCTGCAGATGATTCTGAAC
GGCATTAACAACTATAAAAACCCGAAACTGACCGCGATGCTGACCAAAAA
ATTTTATATGCCGAAAAAAGCGACCGAACTGAAACATCTGCAGTGCCTGG
AAGAAGAACTGAAACCGCTGGAAGAAGTGCTGAACCTGGCGCAGAGCAAA
AACTTTCATTTCGACCCGCGCGATGTGaTTAGCAACATTAACGTGTTTGT
GCTGGAACTGAAAGGCAGCGAAACCACCTTTATGTGCGAATATGCGGATG
AAACCGCGACCATTGTGGAATTTCTGAACCGCTGGATTACCTTTTGCCAG
AGCATTATTAGCACCCTGACC

SEQ ID NO: 10 - IL2_V4 (amino acid) APTSSSTKKTQLQL
EHLLLDLQMILNGINNYKNPKLTAMLTKKFYMPKKATELKHLQCLEEELK
PLEEVLNLAQSKNFIVDPRDVISNINVFVLELKGSETTFMCEYADETATI
VEFLNRWITFCQSIISTLT

SEQ ID NO: 11 - IL2_V5 GCGCCGACCAGCAGCAGCACCAAAAAA
ACCCAGCTGCAGCTGGAACATCTGCTGCTGGATCTGCAGATGATTCTGAA
CGGCATTAACAACTATAAAAACCCGAAACTGACCGCGATGCTGACCAAAA
AATTTTATATGCCGAAAAAAGCGACCGAACTGAAACATCTGCAGTGCCTG
GAAGAAGAACTGAAACCGCTGGAAGAAGTGCTGAACCTGGCGCAGAGCAA
AAACTTTCATTTCGACCCGCGCGATGTGGTTAGCAACATTAACGTGaTTG
TGCTGGAACTGAAAGGCAGCGAAACCACCTTTATGTGCGAATATGCGGAT
GAAACCGCGACCATTGTGGAATTTCTGAACCGCTGGATTACCTTTTGCCA
GAGCATTATTAGCACCCTGACC

SEQ ID NO: 12 - IL2_V5 (amino acid) APTSSSTKKTQLQL
EHLLLDLQMILNGINNYKNPKLTAMLTKKFYMPKKATELKHLQCLEEELK
PLEEVLNLAQSKNFIVDPRDVVSNINVIVLELKGSETTFMCEYADETATI
VEFLNRWITFCQSIISTLT

SEQ ID NO: 13 -IL2_V4_R38A GCGCCGACCAGCAGCAGCACCAA
AAAAACCCAGCTGCAGCTGGAACATCTGCTGCTGGATCTGCAGATGATTC
TGAACGGCATTAACAACTATAAAAACCCGAAACTGACCGCGATGCTGACC
TTCAAATTTTATATGCCGAAAAAAGCGACCGAACTGAAACATCTGCAGTG
CCTGGAAGAAGAACTGAAACCGCTGGAAGAAGTGCTGAACCTGGCGCAGA
GCAAAAACTTTCATTTCGACCCGCGCGATGTGaTTAGCAACATTAACGTG
TTTGTGCTGGAACTGAAAGGCAGCGAAACCACCTTTATGTGCGAATATGC
GGATGAAACCGCGACCATTGTGGAATTTCTGAACCGCTGGATTACCTTTT
GCCAGAGCATTATTAGCACCCTGACC

SEQ ID NO: 14 - IL2_V4_R38A (amino acid) APTSSSTKK
TQLQLEHLLLDLQMILNGINNYKNPKLTAMLTFKFYMPKKATELKHLQCL
EEELKPLEEVLNLAQSKNFIVDPRDVISNINVFVLELKGSETTFMCEYAD
ETATIVEFLNRWITFCQSIISTLT

SEQ ID NO: 15 - IL2_V4_F42A GCGCCGACCAGCAGCAGCACCA
AAAAAACCCAGCTGCAGCTGGAACATCTGCTGCTGGATCTGCAGATGATT
CTGAACGGCATTAACAACTATAAAAACCCGAAACTGACCCGCATGCTGAC
CGCGAAATTTTATATGCCGAAAAAAGCGACCGAACTGAAACATCTGCAGT
GCCTGGAAGAAGAACTGAAACCGCTGGAAGAAGTGCTGAACCTGGCGCAG
AGCAAAAACTTTCATTTTGATCCGCGCGATGTGATTAGCAACATTAACGT
GTTTGTGCTGGAACTGAAAGGCAGCGAAACCACCTTTATGTGCGAATATG
CGGATGAAACCGCGACCATTGTGGAATTTCTGAACCGCTGGATTACCTTT
TGCCAGAGCATTATTAGCACCCTGACC

SEQ ID NO: 16 - IL2_V4_F42A (amino acid) APTSSSTKK
TQLQLEHLLLDLQMILNGINNYKNPKLTRMLTAKFYMPKKATELKHLQCL
EEELKPLEEVLNLAQSKNFIVDPRDVISNINVFVLELKGSETTFMCEYAD
ETATIVEFLNRWITFCQSIISTLT

SEQ ID NO: 17 -IL2_V4_R3SA_F42A GCGCCGACCAGCAGCAGC
ACCAAAAAAACCCAGCTGCAGCTGGAACATCTGCTGCTGGATCTGCAGAT
GATTCTGAACGGCATTAACAACTATAAAAACCCGAAACTGACCGCGATGC
TGACCGCGAAATTTTATATGCCGAAAAAAGCGACCGAACTGAAACATCTG
CAGTGCCTGGAAGAAGAACTGAAACCGCTGGAAGAAGTGCTGAACCTGGC
GCAGAGCAAAAACTTTCATTTCGACCCGCGCGATGTGaTTAGCAACATTA
ACGTGTTTGTGCTGGAACTGAAAGGCAGCGAAACCACCTTTATGTGCGAA
TATGCGGATGAAACCGCGACCATTGTGGAATTTCTGAACCGCTGGATTAC
CTTTTGCCAGAGCATTATTAGCACCCTGACC

SEQ ID NO: 18 - IL2_V4_R38A_F42A (amino acid) APTS
SSTKKTQLQLEHLLLDLQMILNGINNYKNPKLTAMLTAKFYMPKKATELK
HLQCLEEELKPLEEVLNLAQSKNFIVDPRDVISNINVFVLELKGSETTFM
CEYADETATIVEFLNRWITFCQSIISTLT

SEQ ID NO: 19 - Fc (207-230 IMGT allele IGHG1*03,
231- 457 IMGT allele IGHG2*01) AGCAACACTAAAGTCGACA
AGCGAGTAGAACCGAAATCATGCGACAAAACACATACGTGCCCTCCCTGC
CCAGCACCACCTGTCGCGGGCCCCTCTGTTTTCCTGTTTCCACCCAAGCC
AAAGGACACATTGATGATTTCCCGGACTCCTGAAGTCACCTGCGTGGTAG
TAGATGTATCACATGAAGATCCAGAAGTCCAGTTCAACTGGTATGTGGAC
GGAGTAGAGGTACATAATGCCAAGACCAAACCACGGGAAGAGCAGTTCAA
CAGTACTTTCCGGGTAGTTAGCGTTTTGACTGTCGTACACCAAGACTGGC
TTAATGGAAAAGAATACAAGTGTAAGGTAAGCAACAAGGGCCTGCCGGCT
CCGATAGAGAAAACCATTAGCAAGACAAAGGGCCAACCACGCGAACCCCA
GGTATATACCCTCCCACCGTCCCGCGAGGAGATGACTAAGAATCAAGTTT
CTCTCACGTGCTTGGTAAAGGGCTTCTATCCGAGCGATATAGCCGTGGAG
TGGGAGTCTAATGGTCAGCCCGAAAACAATTACAAAACTACGCCTCCTAT
GCTGGACAGTGATGGGAGCTTCTTTCTTTACAGTAAGCTTACCGTGGACA
AGTCTCGGTGGCAACAAGGAAATGTTTTTAGTTGTTCTGTAATGCATGAA
GCACTTCATAACCATTACACCCAGAAAAGTCTGAGCTTGTCCCCGGGAAA
A

SEQ ID NO: 20 - Fc (207-230 IMGT allele IGHG1*03,
231- 457 IMGT allele IGHG2*01)(amino acid) SNTKVDK
RVEPKSCDKTHTCPPCPAPPVAGPSVFLFPPKPKDTLMISRTPEVTCVWD
VSHEDPEVQFNWYVDGVEVHNAKTKPREEQFNSTFRWSVLTWHQDWLNGK
EYKCKVSNKGLPAPIEKTISKTKGQPREPQVYTLPPSREEMTKNQVSLTC
LVKGFYPSDIAVEWESNGQPENNYKTTPPMLDSDGSFFLYSKLTVDKSRW
QQGNVFSCSVMHEALHNHYTQKSLSLSPGK

### Vectors

In some embodiments, nucleic acid constructs described above may be inserted into an expression vector or viral vector by methods known to the art, and nucleic acid molecules may be operably linked to an expression control sequence. Non-limiting examples of expression vectors include plasmid vectors, transposon vectors, cosmid vectors, and viral derived vectors (e.g., any adenoviral derived vectors (AV), cytomegaloviral derived (CMV) vectors, simian viral derived (SV40) vectors, adeno-associated virus (AAV) vectors, lentivirus vectors, and retroviral vectors). In some embodiments, the expression vector is a viral vector.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art.

In some embodiments, nucleic acid molecules are inserted into a vector that is able to express an IL-2 protein of the present disclosure when introduced into an appropriate cell. In some embodiments, the vector includes any of the nucleic acids described herein. In some embodiments, the vector further includes a promoter operationally linked to the nucleic acid. In some embodiments, the promoter is a constitutive promoter. In some embodiments, the promoter is an inducible promoter. In some embodiments, the vector is a viral vector. In some embodiments, the viral vector is a lentiviral vector.

Also provided herein are pharmaceutical compositions that include any of the proteins described herein and a pharmaceutically acceptable carrier. In some embodiments, a pharmaceutical composition includes any of the nucleic acids or any of the vectors described herein. Also provided herein are cells including any of the nucleic acids or any of the vectors described herein. In some embodiments, a pharmaceutical composition includes any of the cells described herein and a pharmaceutically acceptable carrier.

Also provided herein are methods of producing an IL-2 protein, the method including (a) culturing any of the cells described herein in a culture medium under conditions sufficient to express the IL-2 protein; and (b) recovering the IL-2 protein from the cell and/or the culture medium.

### Therapeutic Applications

In some embodiments, the IL-2 proteins or nucleic acid constructs described herein may be used for treating a subject in need thereof. In some embodiments, the subject is diagnosed with an IL-2 associated disease. In some embodiments, the subject is diagnosed with an IL-2 associated cancer. In some embodiments, a pharmaceutical composition that includes an IL-2 protein and a pharmaceutically acceptable carrier can be administered to the subject diagnosed with an IL-2 associated disease. In some embodiments, the pharmaceutical composition can be administered with one or more additional anticancer therapies that include, but are not limited to, ionizing radiation, a chemotherapeutic agent, a therapeutic antibody, and a checkpoint inhibitor.

Interleukin-2 (IL-2) is a cytokine functioning to modify the body’s response to cancer cells, wherein IL-2 helps increase production of different components of the immune system (e.g., T lymphocytes and natural killer (NK) cells). In addition, IL-2 also improves the function of other immune system cells (e.g., lymphokine-activated killer cells and tumor-infiltrating lymphocytes) helping to treat cancer. In some embodiments, IL-2 is used to treat cancers that can include, but are not limited to, renal cell (kidney) cancer, metastatic melanoma, and advanced non-Hodgkin’s lymphomas.

Cancer can refer to a broad group of diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. Cancer or cancer tissue may include a tumor.

Provided herein are methods of treating a cancer in a subject, the method including administering to the subject a therapeutically effective amount of any of the pharmaceutical compositions described herein. In some embodiments, the cancer is carcinoma, lymphoma (e.g., Hodgkin’s and non-Hodgkin’s lymphomas), blastoma, sarcoma, leukemia, squamous cell carcinoma, small cell lung cancer, non-small cell lung cancer, lung adenocarcinoma, squamous cell carcinoma of the lung, peritoneal cancer, hepatocellular carcinoma, gastric cancer, pancreatic cancer, glioma, cervical cancer, ovarian cancer, liver cancer, bladder cancer, breast cancer, colon cancer, colorectal cancer, endometrial or uterine carcinoma, salivary carcinoma, kidney cancer, prostate cancer, vulvar cancer, thyroid cancer, liver carcinoma, other lymphoproliferative disorders, or various types of head and neck cancer. In some embodiments, the cancer is a solid tumor. In some embodiments, the cancer is colorectal cancer. In some embodiments, the subject has previously been administered one or more additional anticancer therapies selected from the group consisting of: ionizing radiation, a chemotherapeutic agent, a therapeutic antibody, and a checkpoint inhibitor. In some embodiments, the subject has been identified or diagnosed as having the cancer.

Cancers suitable for treatment by a method of the present disclosure can include, but are not limited to, bladder cancer, breast cancer, cervical cancer, colon cancer, endometrial cancer, esophageal cancer, fallopian tube cancer, gall bladder cancer, gastrointestinal cancer, head and neck cancer, hematological cancer, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, and prostate cancer. In some embodiments, a cancer for treatment by a method of the present disclosure can include may include, but is not limited to, carcinoma, lymphoma (e.g., Hodgkin’s and non-Hodgkin’s lymphomas), blastoma, sarcoma and leukemia. In some embodiments, cancer may include squamous cell carcinoma, small cell lung cancer, non-small cell lung cancer, lung adenocarcinoma, squamous cell carcinoma of the lung, peritoneal cancer, hepatocellular carcinoma, gastric cancer, pancreatic cancer, glioma, cervical cancer, ovarian cancer, liver cancer, bladder cancer, hepatocellular carcinoma, breast cancer, colon cancer, colorectal cancer, endometrial or uterine carcinoma, salivary carcinoma, kidney cancer, prostate cancer, vulvar cancer, thyroid cancer, liver carcinoma, leukemia and other lymphoproliferative disorders, and various types of head and neck cancer. In some embodiments, the cancer can be an embryonal tumor (Wilms tumor, hepatoblastoma, rhabdoid, neuroblasoma), germ cell tumor (yolk sac tumor, immature teratoma, and embryonal carcinoma), carcinoma (hepatocellular carcinoma and pulmonary squamous cell carcinoma), sarcoma (malignant rhabdoid tumor and RMS), or malignant melanoma.

Also provided herein are methods of increasing memory CD8+ T cells in a subject that include administering to the subject (e.g., any of the subjects having any of the cancers described herein) a therapeutically effective amount of any of the pharmaceutical compositions described herein. In some embodiments, the methods result in about a 1% increase to about a 100% increase, about a 1% increase to about a 80% increase, about a 1% increase to about a 60% increase, about a 1% increase to about a 40% increase, about a 1% increase to about a 20% increase, about a 1% increase to about a 15% increase, about a 1% increase to about a 10% increase, about 1% increase to about a 5% increase, about a 5% increase to about a 100% increase, about a 5% increase to about a 80% increase, about a 5% increase to about a 60% increase, about a 5% increase to about a 40% increase, about a 5% increase to about a 20% increase, about a 5% increase to about a 15% increase, about a 5% increase to about a 10% increase, about a 10% increase to about a 100% increase, about a 10% increase to about a 80% increase, about a 10% increase to about a 60% increase, about a 10% increase to about a 40% increase, about a 10% increase to about a 20% increase, about a 10% increase to about a 15% increase, about a 15% increase to about a 100% increase, about a 15% increase to about a 80% increase, about a 15% increase to about a 60% increase, about a 15% increase to about a 40% increase, about a 15% increase to about a 20% increase, about a 20% increase to about a 100% increase, about a 20% increase to about a 80% increase, about a 20% increase to about a 60% increase, about a 20% increase to about a 40% increase, about a 40% increase to about a 100% increase, about a 40% increase to about a 80% increase, about a 40% increase to about a 60% increase, about a 60% increase to about a 100% increase, about a 60% increase to about a 80% increase, or about a *0% increase to about a 100% increase, in memory CD8+ T cells (e.g., as measured as described in the Examples section), e.g., as compared to the level of memory CD8+ T cells prior to the administering.

Also provided herein are methods of increasing CD8+ T cells in a subject that include administering to the subject (e.g., any of the subjects having any of the cancers described herein) a therapeutically effective amount of any of the pharmaceutical compositions described herein. In some embodiments, the methods result in about a 1% increase to about a 100% increase, about a 1% increase to about a 80% increase, about a 1% increase to about a 60% increase, about a 1% increase to about a 40% increase, about a 1% increase to about a 20% increase, about a 1% increase to about a 15% increase, about a 1% increase to about a 10% increase, about 1% increase to about a 5% increase, about a 5% increase to about a 100% increase, about a 5% increase to about a 80% increase, about a 5% increase to about a 60% increase, about a 5% increase to about a 40% increase, about a 5% increase to about a 20% increase, about a 5% increase to about a 15% increase, about a 5% increase to about a 10% increase, about a 10% increase to about a 100% increase, about a 10% increase to about a 80% increase, about a 10% increase to about a 60% increase, about a 10% increase to about a 40% increase, about a 10% increase to about a 20% increase, about a 10% increase to about a 15% increase, about a 15% increase to about a 100% increase, about a 15% increase to about a 80% increase, about a 15% increase to about a 60% increase, about a 15% increase to about a 40% increase, about a 15% increase to about a 20% increase, about a 20% increase to about a 100% increase, about a 20% increase to about a 80% increase, about a 20% increase to about a 60% increase, about a 20% increase to about a 40% increase, about a 40% increase to about a 100% increase, about a 40% increase to about a 80% increase, about a 40% increase to about a 60% increase, about a 60% increase to about a 100% increase, about a 60% increase to about a 80% increase, or about a *0% increase to about a 100% increase, in CD8+ T cells (e.g., as measured as described in the Examples section), e.g., as compared to the level of CD8+ T cells prior to the administering.

Also provided herein are methods of decreasing Treg cells in a solid tumor in a subject that include administering to the subject (e.g., any of the subjects having any of the cancers described herein) a therapeutically effective amount of any of the pharmaceutical compositions described herein. In some embodiments, the methods result in about a 1% decrease to about a 99% decrease, about a 1% decrease to about a 80% decrease, about a 1% decrease to about a 60% decrease, about a 1% decrease to about a 40% decrease, about a 1% decrease to about a 20% decrease, about a 1% decrease to about a 15% decrease, about a 1% decrease to about a 10% decrease, about a 1% decrease to about a 5% decrease, about a 5% decrease to about a 99% decrease, about a 5% decrease to about a 80% decrease, about a 5% decrease to about a 60% decrease, about a 5% decrease to about a 40% decrease, about a 5% decrease to about a 20% decrease, about a 5% decrease to about a 15% decrease, about a 5% decrease to about a 10% decrease, about a 10% decrease to about a 99% decrease, about a 10% decrease to about a 80% decrease, about a 10% decrease to about a 60% decrease, about a 10% decrease to about a 40% decrease, about a 10% decrease to about a 20% decrease, about a 10% decrease to about a 15% decrease, about a 15% decrease to about a 99% decrease, about a 15% decrease to about a 80% decrease, about a 15% decrease to about a 60% decrease, about a 15% decrease to about a 40% decrease, about a 15% decrease to about a 20% decrease, about a 20% decrease to about a 99% decrease, about a 20% decrease to about a 80% decrease, about a 20% decrease to about a 60% decrease, about a 20% decrease to about a 40% decrease, about a 40% decrease to about a 99% decrease, about a 40% decrease to about a 80% decrease, about a 40% decrease to about a 60% decrease, about a 60% decrease to about a 99% decrease, about a 60% decrease to about a 80% decrease, about a 80% decrease to about a 99% decrease, in the level of Treg cells in a solid tumor in a subject, e.g., as compared to the level of Treg cells in the solid tumor in the subject prior to the administering. Non-limiting methods of detecting Treg cells are described in the Examples.

Also provided herein are methods of decreasing a rate of growth of a solid tumor in a subject that include administering to the subject (e.g., any of the subjects having any of the cancers described herein) a therapeutically effective amount of any of the pharmaceutical compositions described herein. In some embodiments, the methods result in about a 1% decrease to about a 99% decrease, about a 1% decrease to about a 80% decrease, about a 1% decrease to about a 60% decrease, about a 1% decrease to about a 40% decrease, about a 1% decrease to about a 20% decrease, about a 1% decrease to about a 15% decrease, about a 1% decrease to about a 10% decrease, about a 1% decrease to about a 5% decrease, about a 5% decrease to about a 99% decrease, about a 5% decrease to about a 80% decrease, about a 5% decrease to about a 60% decrease, about a 5% decrease to about a 40% decrease, about a 5% decrease to about a 20% decrease, about a 5% decrease to about a 15% decrease, about a 5% decrease to about a 10% decrease, about a 10% decrease to about a 99% decrease, about a 10% decrease to about a 80% decrease, about a 10% decrease to about a 60% decrease, about a 10% decrease to about a 40% decrease, about a 10% decrease to about a 20% decrease, about a 10% decrease to about a 15% decrease, about a 15% decrease to about a 99% decrease, about a 15% decrease to about a 80% decrease, about a 15% decrease to about a 60% decrease, about a 15% decrease to about a 40% decrease, about a 15% decrease to about a 20% decrease, about a 20% decrease to about a 99% decrease, about a 20% decrease to about a 80% decrease, about a 20% decrease to about a 60% decrease, about a 20% decrease to about a 40% decrease, about a 40% decrease to about a 99% decrease, about a 40% decrease to about a 80% decrease, about a 40% decrease to about a 60% decrease, about a 60% decrease to about a 99% decrease, about a 60% decrease to about a 80% decrease, about a 80% decrease to about a 99% decrease, in the rate of growth of a solid tumor in a subject, e.g., as compared to the rate of growth of the solid tumor prior to the administering. In some embodiments, the rate of growth of a solid tumor can be determined using multiple assessments of solid tumor volume over time using magnetic resonance imaging or computed tomography.

Also provided herein are methods of decreasing a volume of a solid tumor in a subject that include administering to the subject (e.g., any of the subjects having any of the cancers described herein) a therapeutically effective amount of any of the pharmaceutical compositions described herein. In some embodiments, the methods result in about a 1% decrease to about a 99% decrease, about a 1% decrease to about a 80% decrease, about a 1% decrease to about a 60% decrease, about a 1% decrease to about a 40% decrease, about a 1% decrease to about a 20% decrease, about a 1% decrease to about a 15% decrease, about a 1% decrease to about a 10% decrease, about a 1% decrease to about a 5% decrease, about a 5% decrease to about a 99% decrease, about a 5% decrease to about a 80% decrease, about a 5% decrease to about a 60% decrease, about a 5% decrease to about a 40% decrease, about a 5% decrease to about a 20% decrease, about a 5% decrease to about a 15% decrease, about a 5% decrease to about a 10% decrease, about a 10% decrease to about a 99% decrease, about a 10% decrease to about a 80% decrease, about a 10% decrease to about a 60% decrease, about a 10% decrease to about a 40% decrease, about a 10% decrease to about a 20% decrease, about a 10% decrease to about a 15% decrease, about a 15% decrease to about a 99% decrease, about a 15% decrease to about a 80% decrease, about a 15% decrease to about a 60% decrease, about a 15% decrease to about a 40% decrease, about a 15% decrease to about a 20% decrease, about a 20% decrease to about a 99% decrease, about a 20% decrease to about a 80% decrease, about a 20% decrease to about a 60% decrease, about a 20% decrease to about a 40% decrease, about a 40% decrease to about a 99% decrease, about a 40% decrease to about a 80% decrease, about a 40% decrease to about a 60% decrease, about a 60% decrease to about a 99% decrease, about a 60% decrease to about a 80% decrease, about a 80% decrease to about a 99% decrease, in the volume of a solid tumor in a subject, e.g., as compared to the volume of the solid tumor prior to the administering. In some embodiments, the volume of a solid tumor can be determined using magnetic resonance imaging or computed tomography.

### EXAMPLES

The disclosure is further described in the following examples, which do not limit the scope of the disclosure described in the claims.

### Example 1― Variants of IL-2

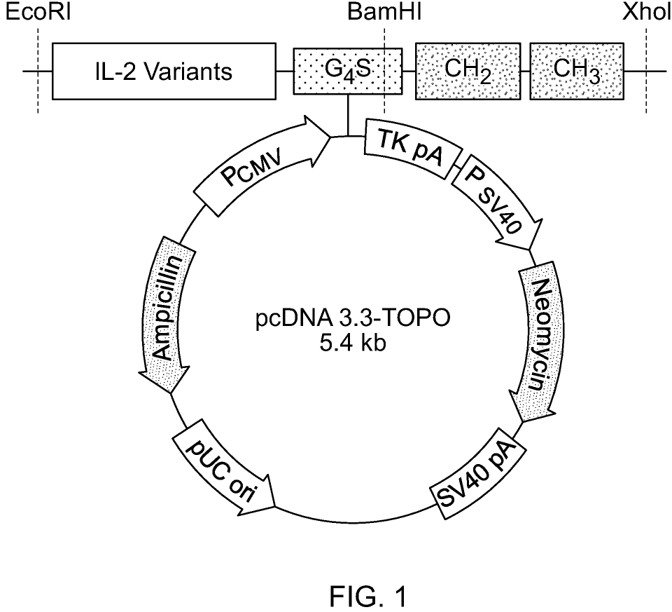

IL-2 proteins were produced using an IL2-G4S linker-Fc_pcDNA3.3 plasmid. The sequences of wild-type IL-2 and variants of IL-2 protein are listed in Table 1. Specifically, the IL-2 protein was produced using an animal cell expression vector pcDNA3.3 wherein restriction enzymes EcoRI and BamHI were inserted at restriction enzyme sites. Human IL-2 signal peptide (UniPortKB: P60568) was used for the signal peptide (1aa - 20aa of P60568) and 207aa - 230aa IMGT allele IGHG1*03, 231aa - 457aa IMGT allele IGHG2*01 was used for the Fc region. A DNA construct encoding an IL-2 protein is shown in FIG. 1.

TABLE 1

| | |
|---|---|
| Name | Sequence |
| supMD | R38A, F42K, L80F, R81D, L85V, I86V, I92F |
| IL2_V1 | R38A, F42K, R81D, L85V, I86V, I92F |
| IL2_V2 | R38A, F42K, L80F, L85V, I86V, I92F |
| IL2_V3 | R38A, F42K, L80F, R81D, I86V, I92F |
| IL2_V4 | R38A, F42K, L80F, R81D, L85V, I92F |
| IL2_V5 | R38A, F42K, L80F, R81D, L85V, I86V |

### Example 2 ―Analyzing and Characterization of IL-2 Variants

IL-2 variants were inserted into plasmids and used to produce IL-2 proteins using Expi293 expression system (Invitrogen). The proteins were then purified using AktaPure (GE healthcare), AktaPrime purifier (GE healthcare), and MabselectSURE column (GE healthcare, Cat#11-0034-95). The purified proteins were run through a desalting column (GE healthcare, Cat#17-1408-01) and protein concentration was measures using Multiskan GO (Thermo). Results are shown in Table 2.

TABLE 2

| IL-2 type | Concentration (mg/mL) | Volume (mL) | Yield (mg) | Production ratio (Mutant/SupMD) |
|---|---|---|---|---|
| SupMD | 2.32 | 4.5 | 10.44 | 1 |
| IL2_V1 | 1.32 | 4.5 | 5.94 | 0.569 |
| IL2_V2 | 0.78 | 4.5 | 3.52 | 0.337 |
| IL2_V3 | 1.58 | 4.5 | 7.13 | 0.683 |
| IL2_V4 | 1.88 | 4.5 | 8.46 | 0.810 |
| IL2_V5 | 2.54 | 4.5 | 11.43 | 1.095 |

### Example 3 - Analyzing Proteins With SDS-PAGE

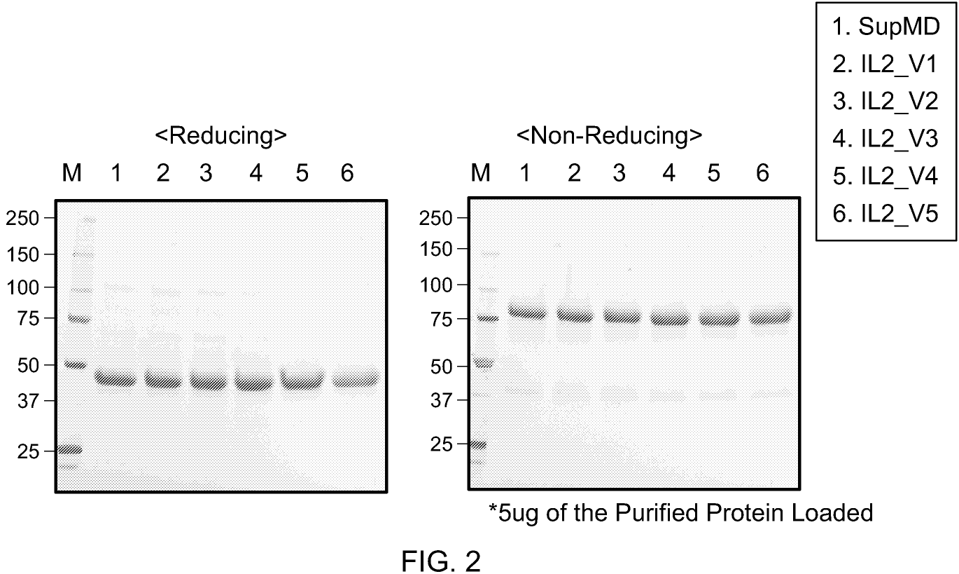

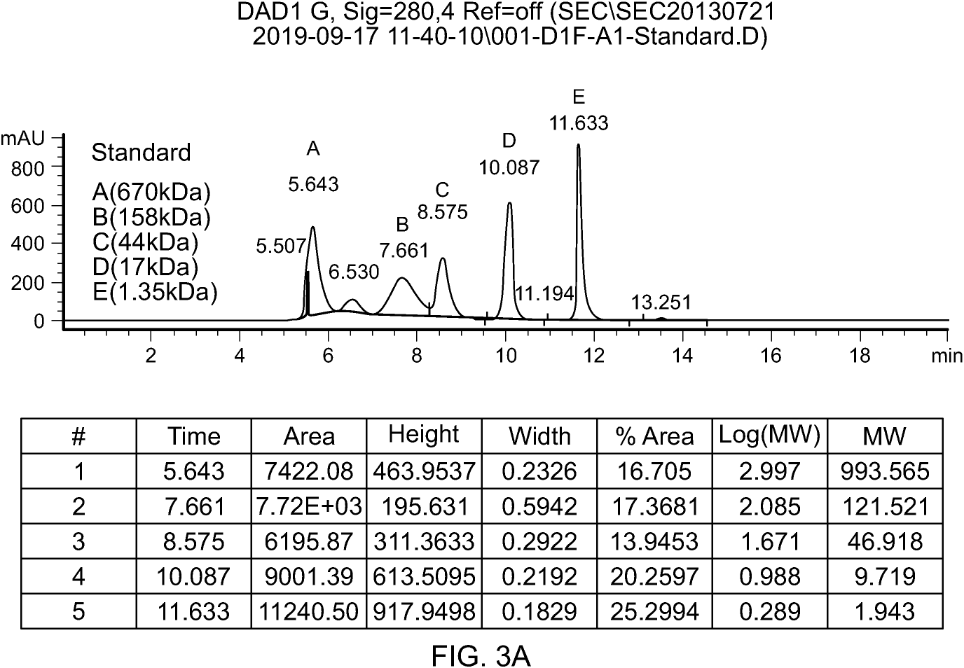

The IL-2 variants were added to LDS sample buffer (Invitrogen, Cat#B0007), wherein a sample reducing agent (Invitrogen, Cat#B0004) was added to the reducing condition group and incubated for 10 minutes at 70° C. SDS running buffer (Bio-rad, Cat#1610732) was added to the prepared samples and the samples were run for 30 minutes using Mini-PROTEIN TGX Stain-Free Gel (Bio-rad, Cat#456-8096). Results were analyzed using Chemidoc (Bio-rad) (FIG. 2).

### Example 4 - Analyzing Affinity to IL-2 Receptors

The affinity of IL-2 variants to IL-2 receptors, IL2-RA and IL2-RB, was analyzed using surface plasmon resonance (SPR). The IL-2 variants were diluted to a concentration of 2 ug/mL then immobilized on CM5 chip (GE Healthcare, Cat#BR-1005-30). The receptors IL-2RA or IL-2RB (Sino) were injected at concentrations 100, 50, 25, 12.5, 6.25, 3.125 nM with an association time of 150 seconds and dissociation time of 240 seconds. Biacore T200 (GE healthcare) was used to measure and analyze affinity of each IL-2 variant, where the results were analyzed with a 1:1 binding model by BIAevaluation software. Results show affinity of IL2_V4 to IL2-RB is maintained to be closest to that of wild-type IL-2 (Table 3, Table 4).

TABLE 3

Affinity to IL2-RA

| | | | |
|---|---|---|---|
| IL-2 type | ka (1/Ms) | kd(1/s) | KD (M) |
| SupMD | Low binding | | |
| IL2_V1 | | | |
| IL2_V2 | | | |
| IL2_V3 | | | |
| IL2_V4 | | | |
| IL2_V5 | | | |

TABLE 4

Affinity to IL2-RB

| IL-2 type | ka (1/Ms) | kd (1/s) | KD (M) |
|---|---|---|---|
| SupMD | 1.358E+5 | 0.004004 | 2.948E-8 |
| IL2_V1 | 1.741E+5 | 0.01159 | 6.657E-8 |
| IL2_V2 | 1.278E+5 | 0.01582 | 1.239E-7 |
| IL2_V3 | 8.244E+4 | 0.01794 | 2.176E-7 |
| IL2_V4 | 1.760E+5 | 0.005111 | 2.903E-8 |
| IL2_V5 | 1.220E+5 | 0.009359 | 7.670E-8 |

### Example 5 - Size Exclusion Chromatography

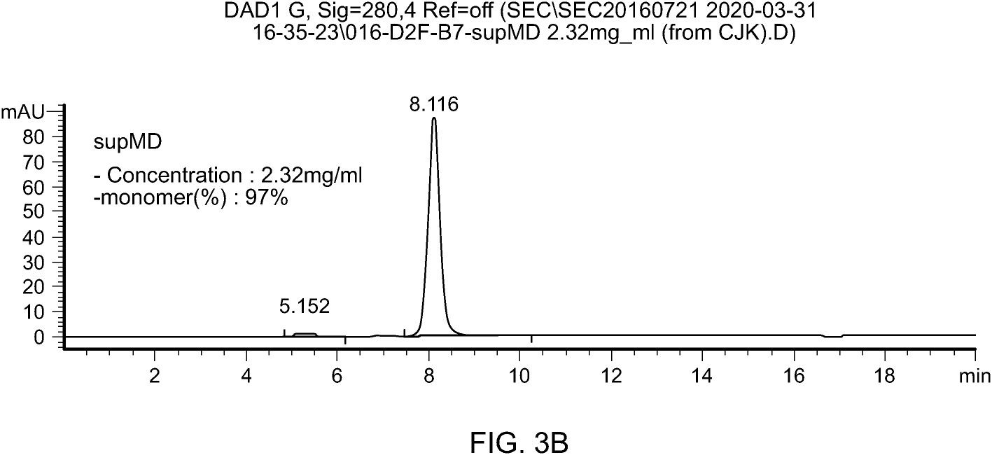

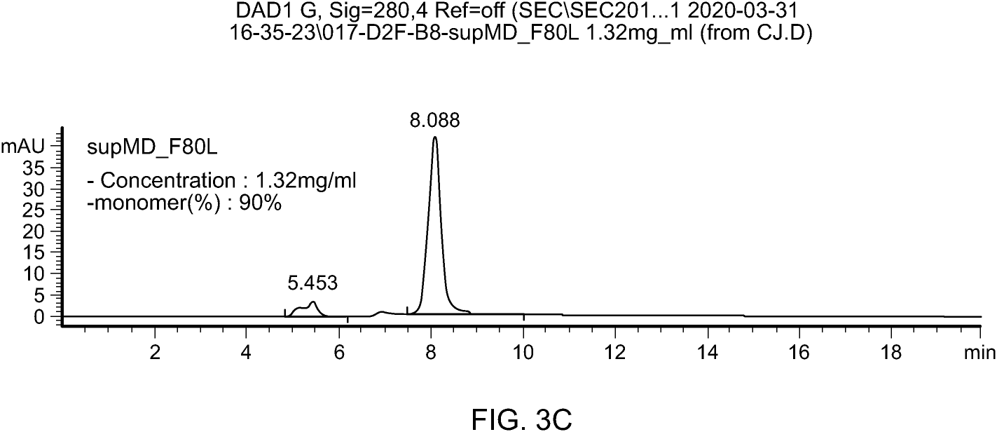

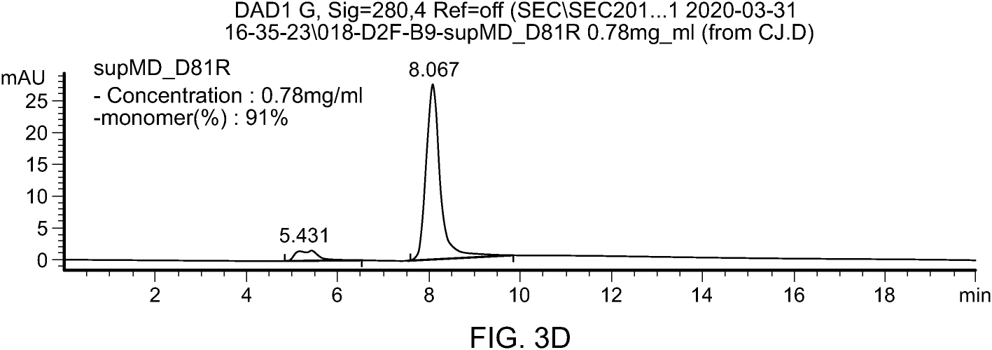

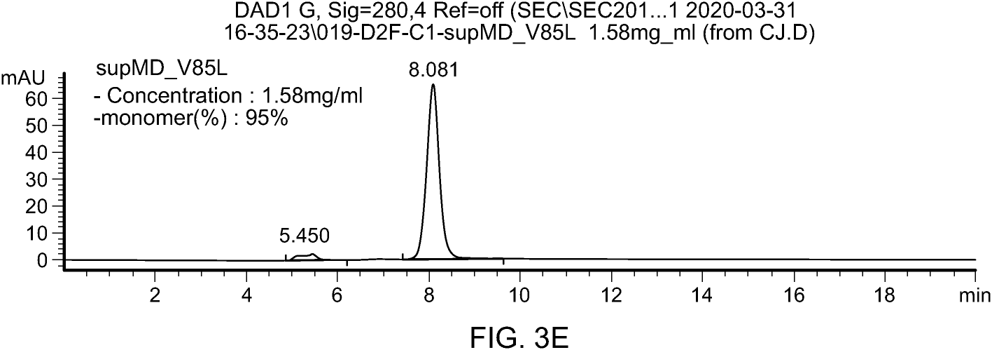

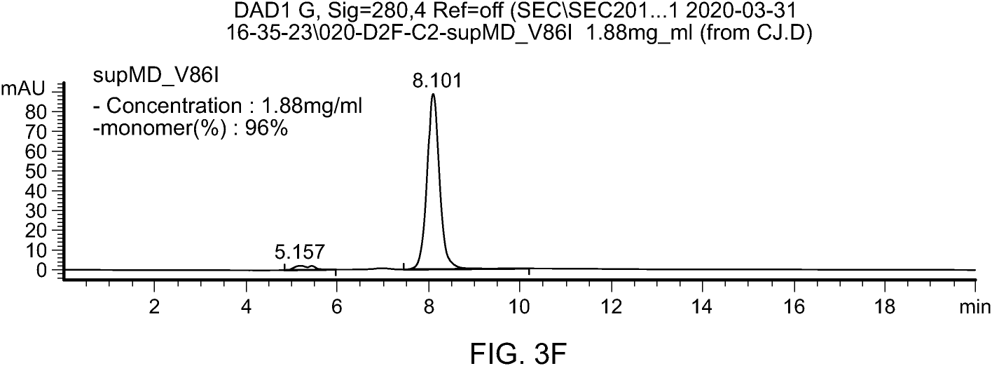

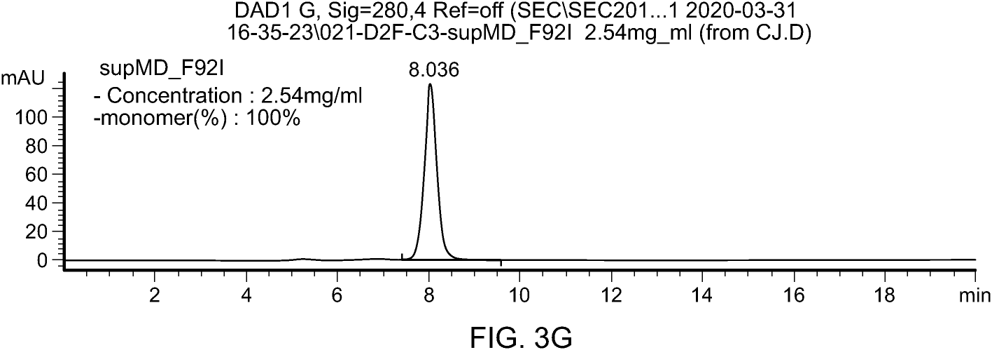

The IL-2 variants were analyzed using HPLC( Agilent Technologies, 1260 infinity II LC system) and size exclusion column(Tosoh, TSKgel G3000 SWXL, 7.8 × 300 mm, Part No.0008541, Column No.004E04320E). An eluent including PBS pH 7.4 was used in the mobile phase while running the HPLC instrument. Gel filtration standard (BIO-RAD, Cat. #151-1901) was used for the control (FIGS. 3A-3G).

### Example 6 - Additional IL-2 Variants From IL2_V4

Additional IL-2 variants were produced as described in Example 1. The sequences of the additional IL-2 variants are listed in Table 5.

TABLE 5

| | |
|---|---|
| Name | Mutation |
| IL2_V4_R38A | R38A, L80F, R81D, L85V, I92F |
| IL2_V4_F42A | F42A, L80F, R81D, L85V, I92F |
| IL2_V4_R38A, F42A | R38A, F42A, L80F, R81D, L85V, I92F |

### Example 7 - Analyzing and Characterization of Additional IL-2 Variants

Additional IL-2 variants were inserted into plasmids and used to produce IL-2 proteins using Expi293 expression system (Invitrogen). The proteins were analyzed and characterized as described in Example 2. Results are shown in Table 6.

TABLE 6

| IL-2 type 1 | Concentration (mg/mL) | Volume | Yield (mg) | Production ratio (Mutant/SupMD) |
|---|---|---|---|---|
| IL2 wt | 0.15 | 2.5 | 0.37 | 1 |
| IL2_V4 | 0.28 | 3 | 0.85 | 2.297 |
| IL2_V4_R38A | 0.30 | 2 | 0.60 | 1.621 |
| IL2_V4_F42A | 0.38 | 4 | 1.52 | 4.108 |
| IL2_V4_R38A_F42A | 0.23 | 4 | 0.92 | 2.486 |

### Example 8 - Analyzing Proteins With SDS-PAGE

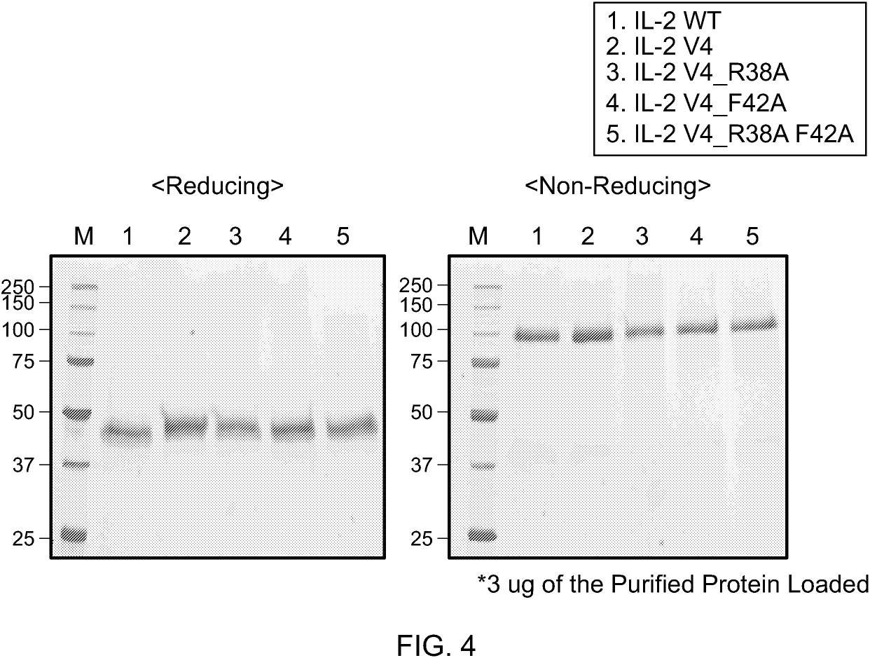

The additional IL-2 variants were added to LDS sample buffer (Invitrogen, Cat#B0007), wherein a sample reducing agent (Invitrogen, Cat#B0004) was added to the reducing condition group and incubated for 10 minutes at 70° C. SDS running buffer (Bio-rad, Cat#1610732) was added to the prepared samples and the samples were run for 30 minutes using Mini-PROTEIN TGX Stain-Free Gel (Bio-rad, Cat#456-8096) as described in Example 3. Results were analyzed using Chemidoc (Bio-rad) (FIG. 4).

### Example 9 - Analyzing Affinity to IL-2 Receptors

The affinity of additional IL-2 variants to IL-2 receptors, IL2-RA and IL2-RB, was analyzed using surface plasmon resonance (SPR). The IL-2 variants were diluted to a concentration of 2 ug/mL then immobilized on CM5 chip (GE Healthcare, Cat#BR-1005-30). Biacore T200 (GE healthcare) was used to measure and analyze affinity of each IL-2 variant, where the results were analyzed with a 1:1 binding model by BIAevaluation software, as described in Example 4. Results are shown in Table 7 and Table 8.

TABLE 7

Affinity to IL2-RA

| IL-2 type | ka (1/Ms) | kd (1/s) | KD (M) |
|---|---|---|---|
| IL2 wt | 2.829E+5 | 2.581E-4 | 9.122E-10 |
| IL2_V4 | - | - | - |
| IL2_V4_R38A | 6.851E+5 | 0.002268 | 3.310E-9 |
| IL2_V4_F42A | 1.170E+5 | 7.894E-4 | 6.745E-9 |
| IL2_V4_R38A_F42A | 4557 | 0.005624 | 1.234E-6 |

TABLE 8

Affinity to IL2-RB

| IL-2 type | ka (1/Ms) | kd (1/s) | KD (M) |
|---|---|---|---|
| IL2 wt | 4.209E+5 | 0.02645 | 6.284E-8 |
| IL2_V4 | 5.916E+5 | 0.005320 | 8.993E-9 |
| IL2_V4_R38A | 7.587E+5 | 0.005557 | 7.325E-9 |
| IL2_V4_F42A | 5.220E+5 | 0.004921 | 9.428E-9 |
| IL2_V4_R38A_F42A | 6.712E+5 | 0.005035 | 7.502E-9 |

### Example 10 - Size Exclusion Chromatography

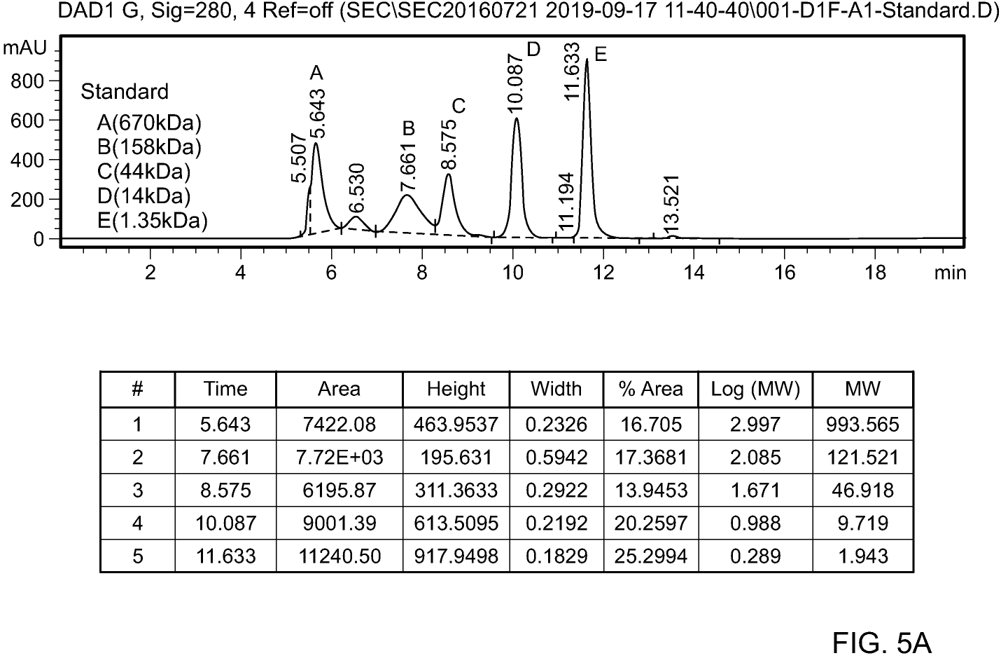

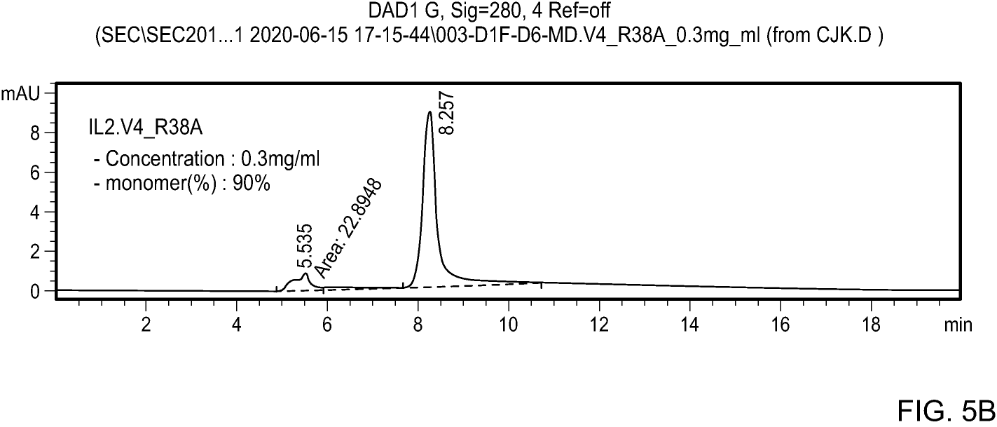

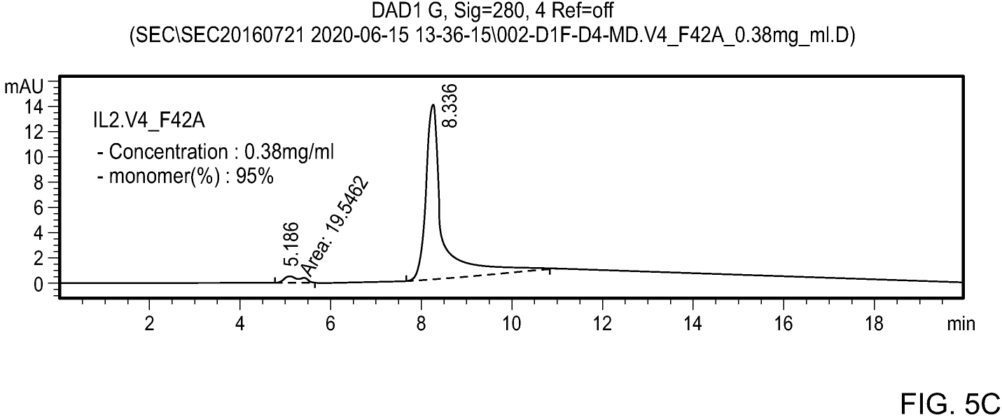

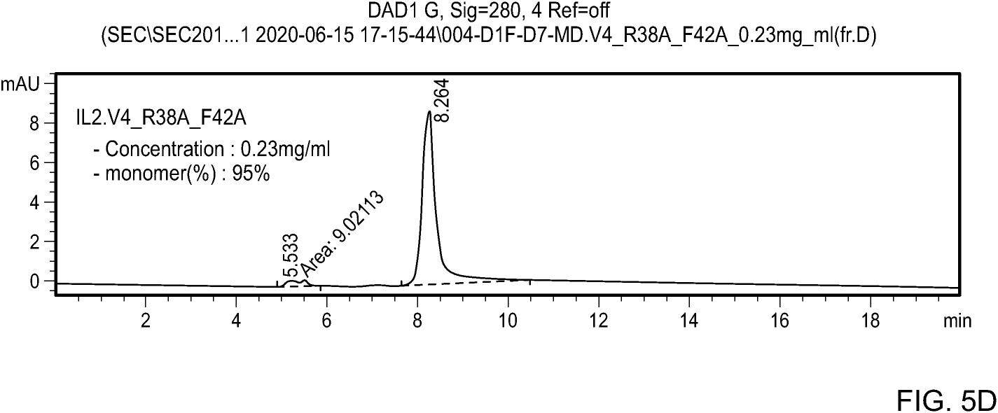

The additional IL-2 variants were analyzed using HPLC( Agilent Technologies, 1260 infinity II LC system) and size exclusion column (Tosoh, TSKgel G3000 SWXL, 7.8 × 300 mm, Part No.0008541, Column No.004E04320E), where an eluent including PBS pH 7.4 was used in the mobile phase while running the HPLC instrument, as described in Example 5. Gel filtration standard (BIO-RAD, Cat. #151-1901) was used for the control (FIGS. 5A-5D).

### Example 11 ― Analysis of Efficacy of IL-2 Variants in in Vivo Mouse Model

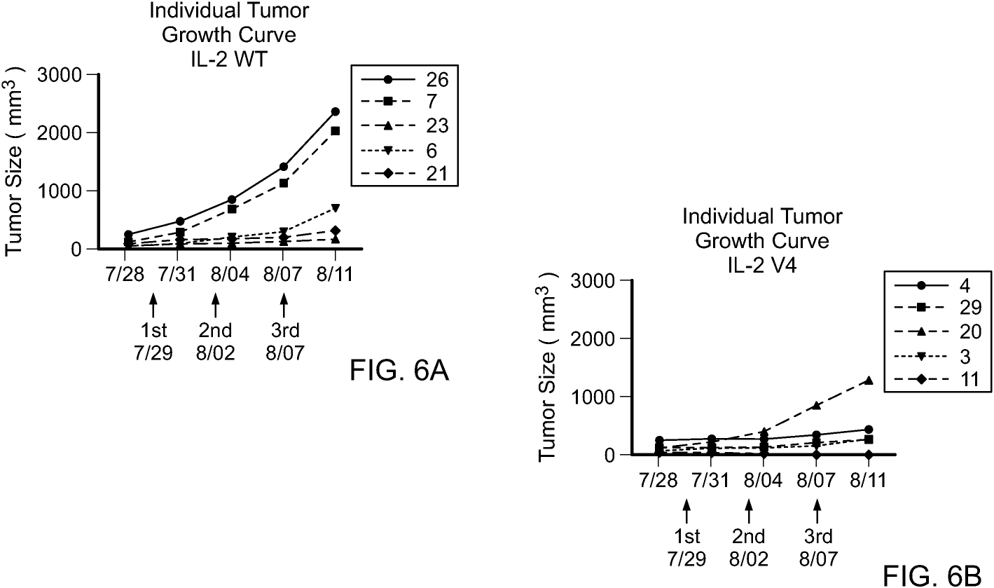

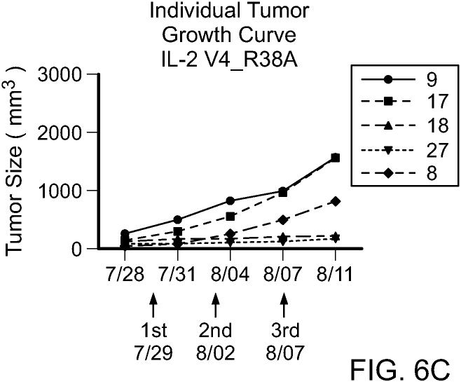

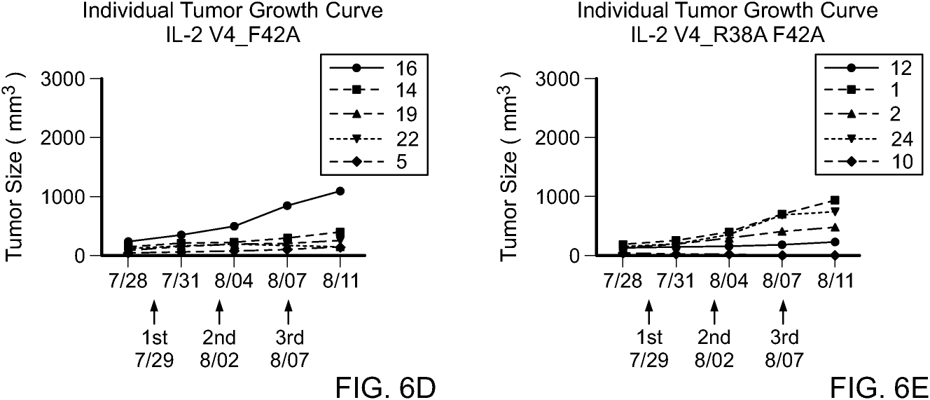

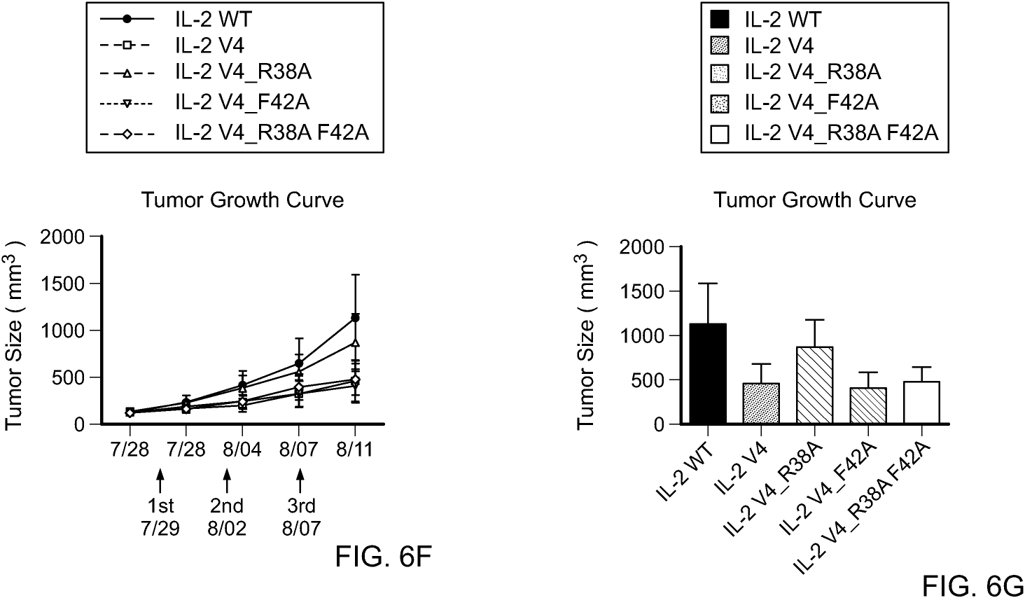

MC38 cell line cells from C57/BL/6 murin colon cancer cells were subcutaneously injected into C57BL/6 mice (0.5 × 10⁶ cells/head) and after about 2 weeks, once the tumor reached a size that was visible to the naked eye, the size of the tumors were measured using TM900 tumor auto-calculator software (FIGS. 6A-6G).

The mice were divided into 5 groups which would receive treatment with the IL2 variants, IL-2 WT, IL-2 V4, IL-2 V4_R38A, IL-2 V4_F42A, and IL-2 V4_R3 8A/F42A. The IL2 mutant proteins were injected (20 µg/head) on day 1, day 5, and day 10 for a total of 3 injections, 2 weeks after tumor inoculation.

Retro-orbital blood was collected from the mice 4 days after the last injection. At the time of blood collection, 100 µl of blood was collected and 50 µl was placed in a 5 mL FACS tube, and treated with fluorescent antibodies for 30 minutes. 1.5 mL of 1x RBC lysis buffer was added and incubated for 10 minutes. After centrifugation for 4 minutes at 2,000 rpm using a centrifuge, all supernatant was discarded. 2 mL of FACS wash buffer was added to the tube and centrifugation was performed for 4 min at 2,000 rpm. This process was then repeated two more times and the sample was analyzed using FACS.

### Analysis of Mouse Regulatory T Cells (T_{reg})

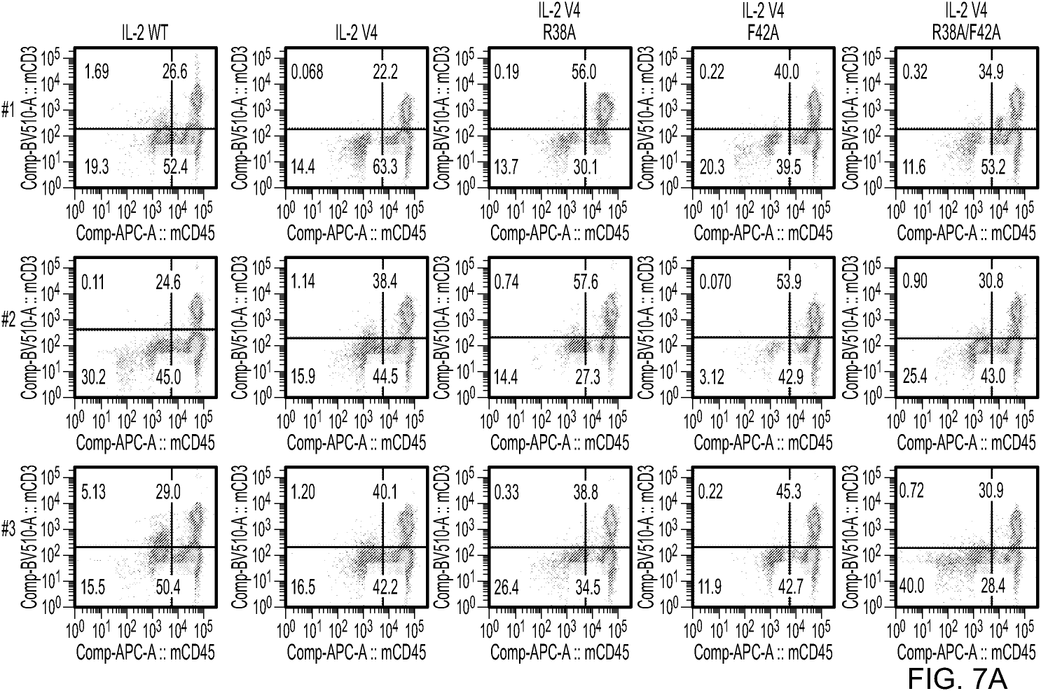

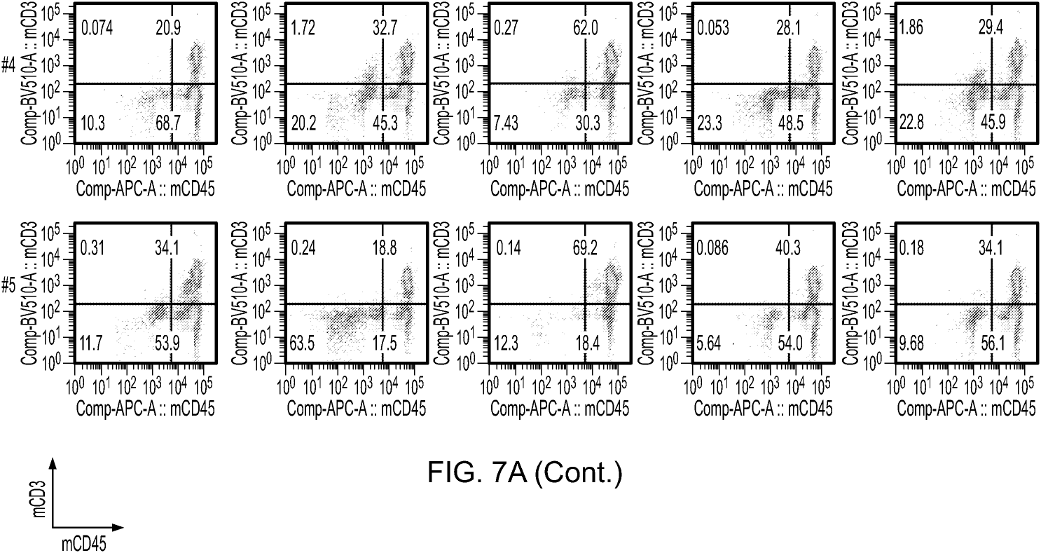

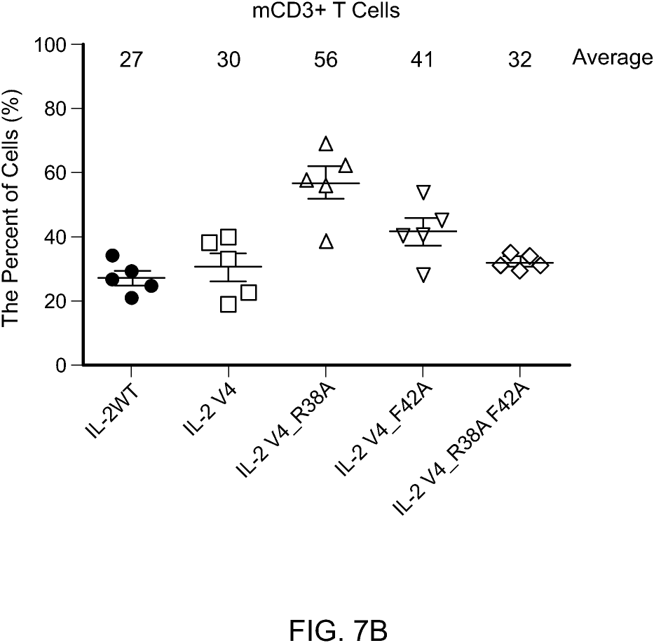

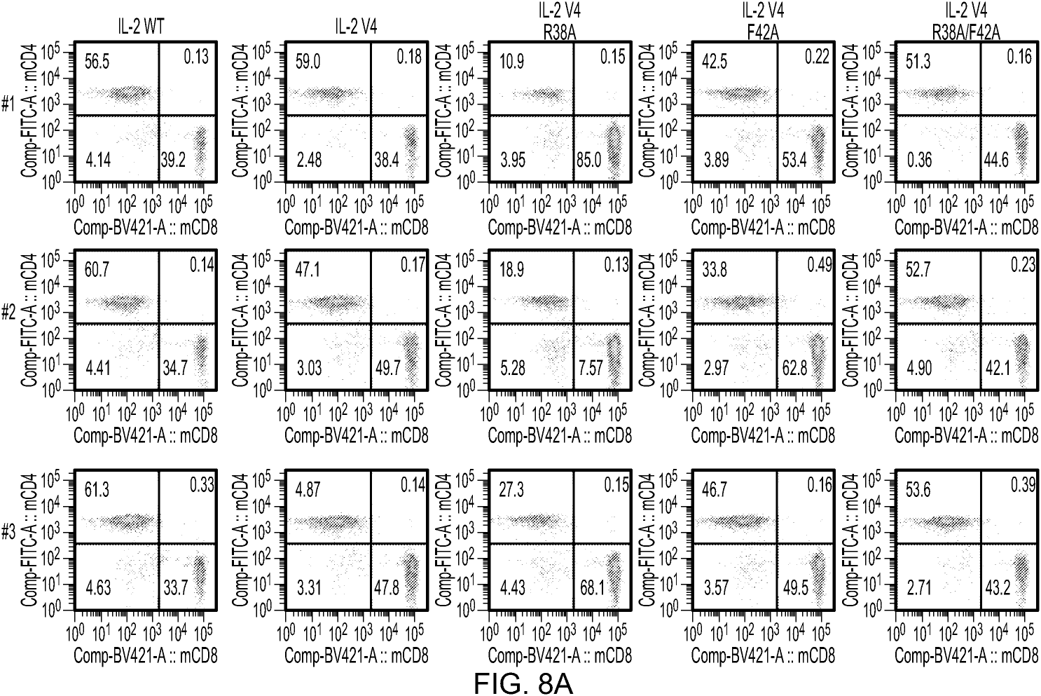

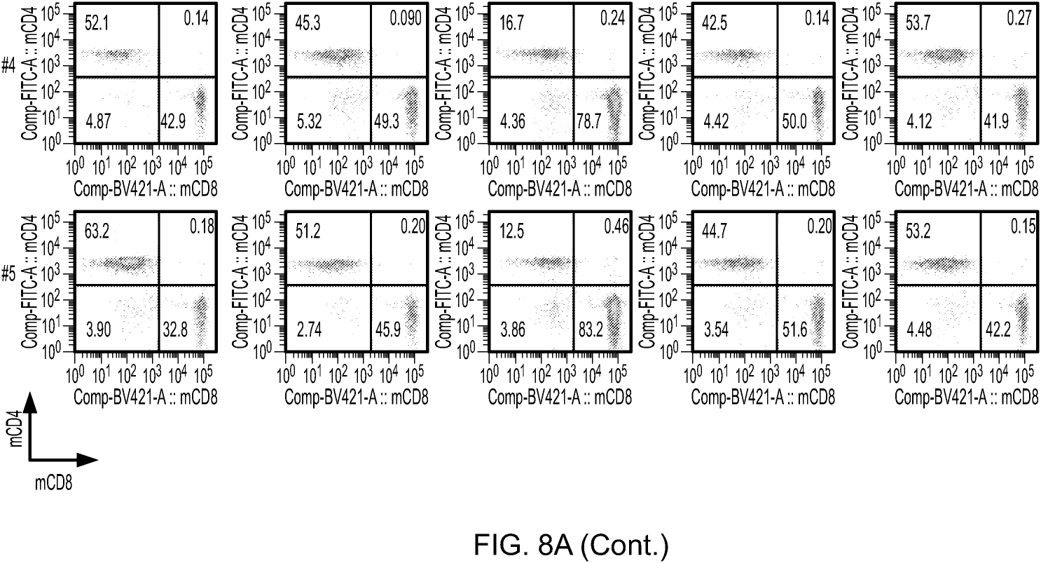

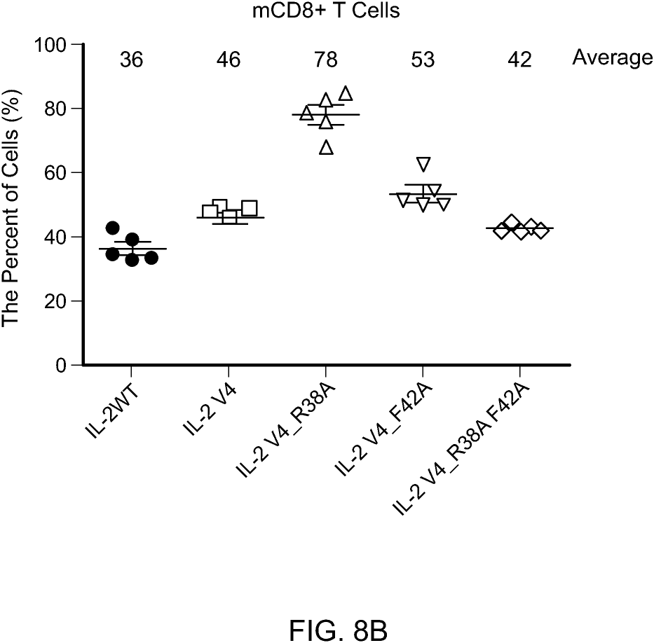

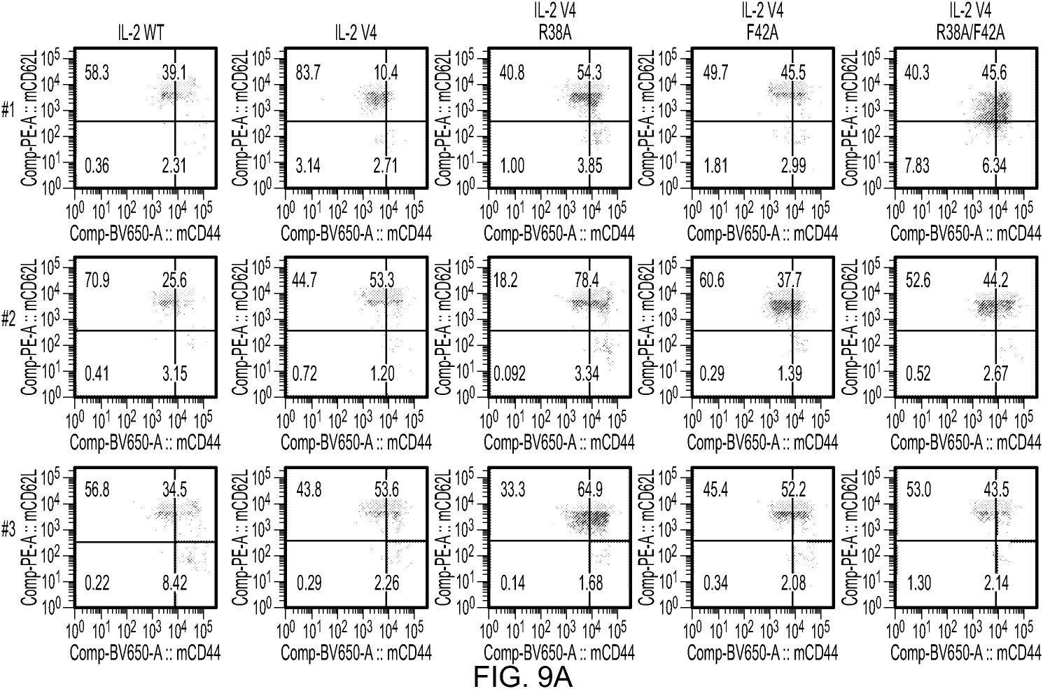

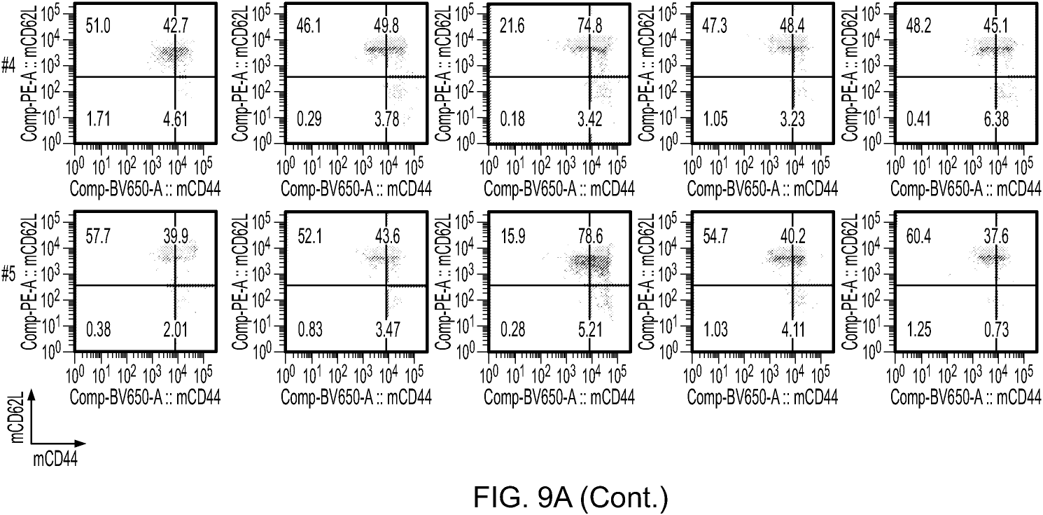

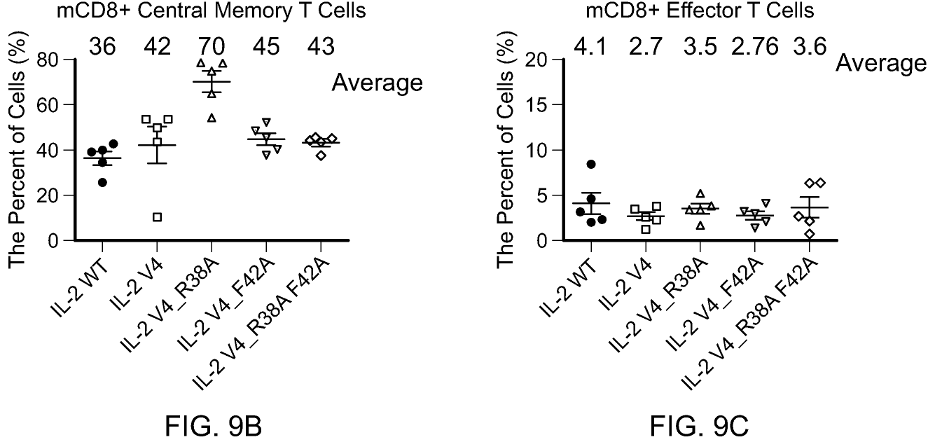

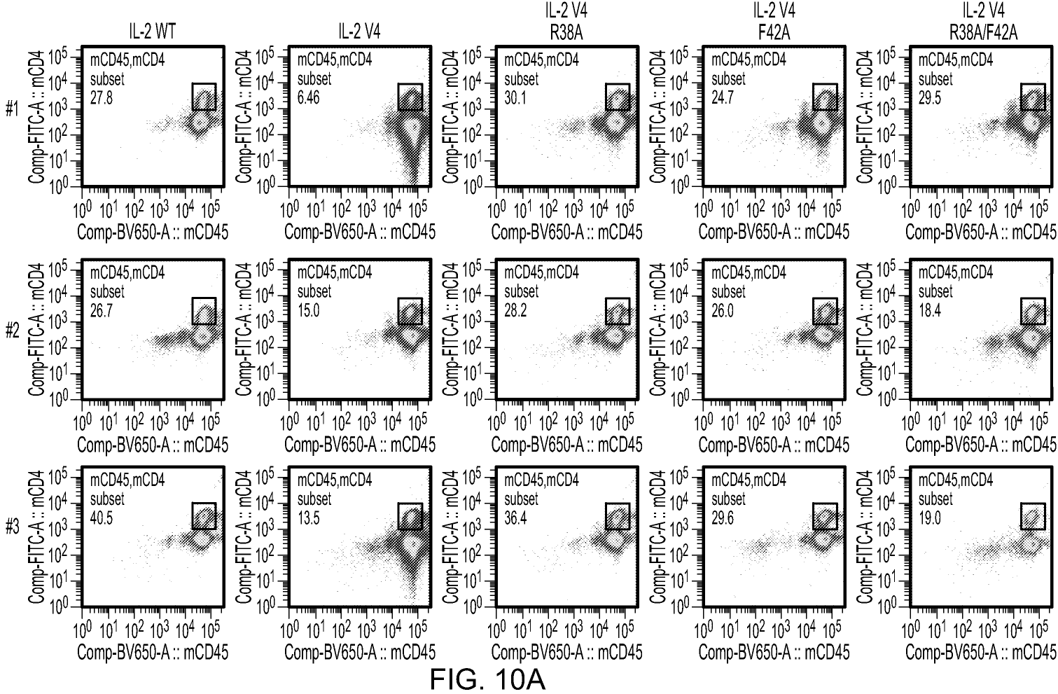

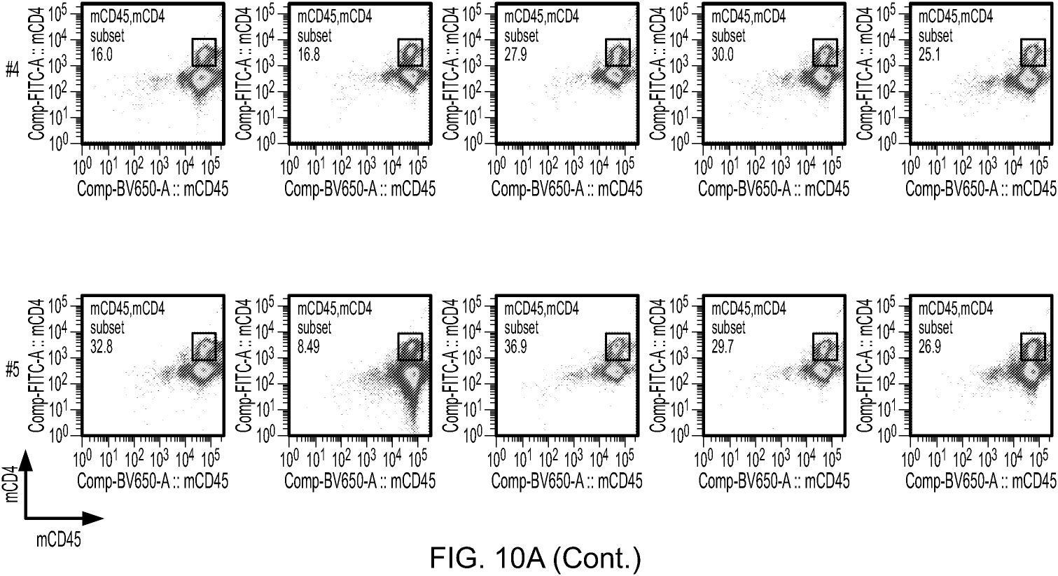

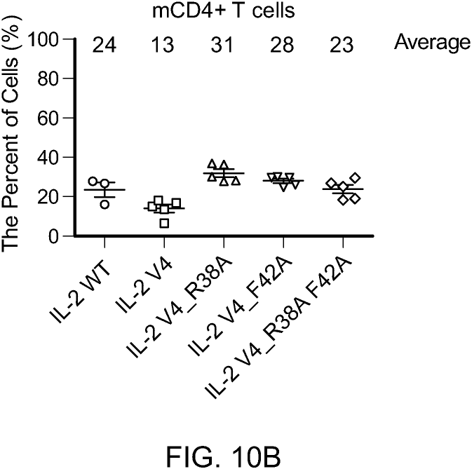

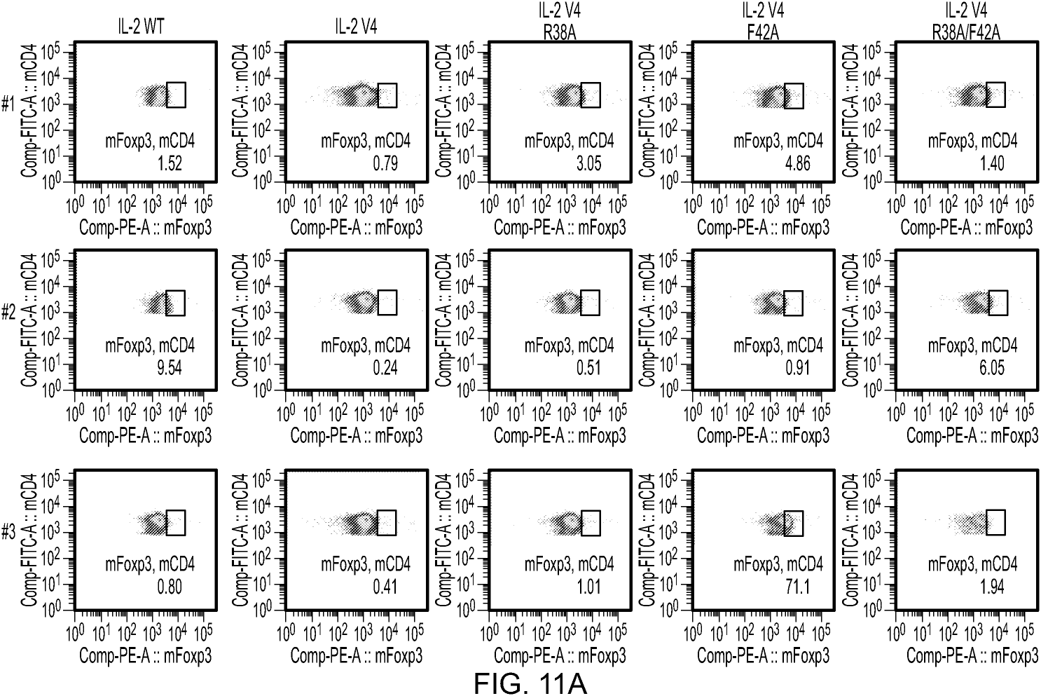

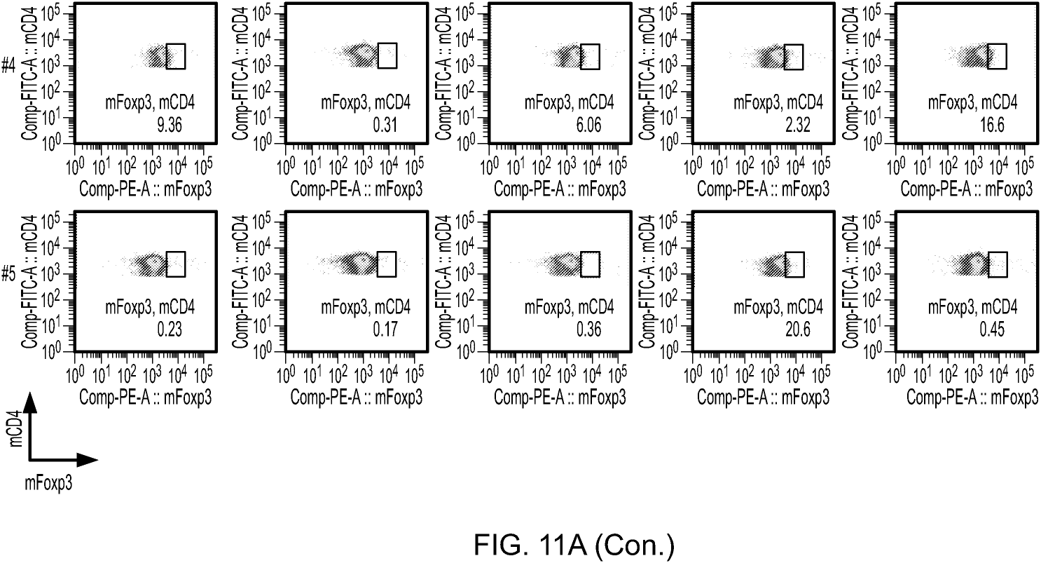

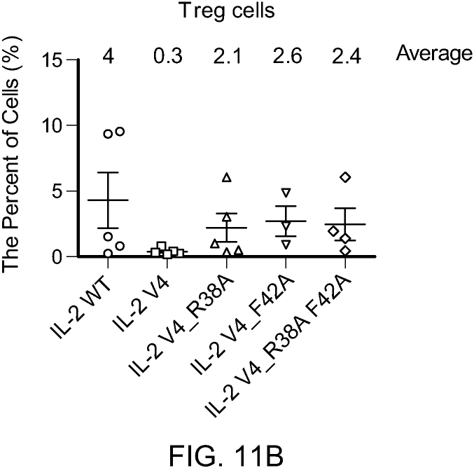

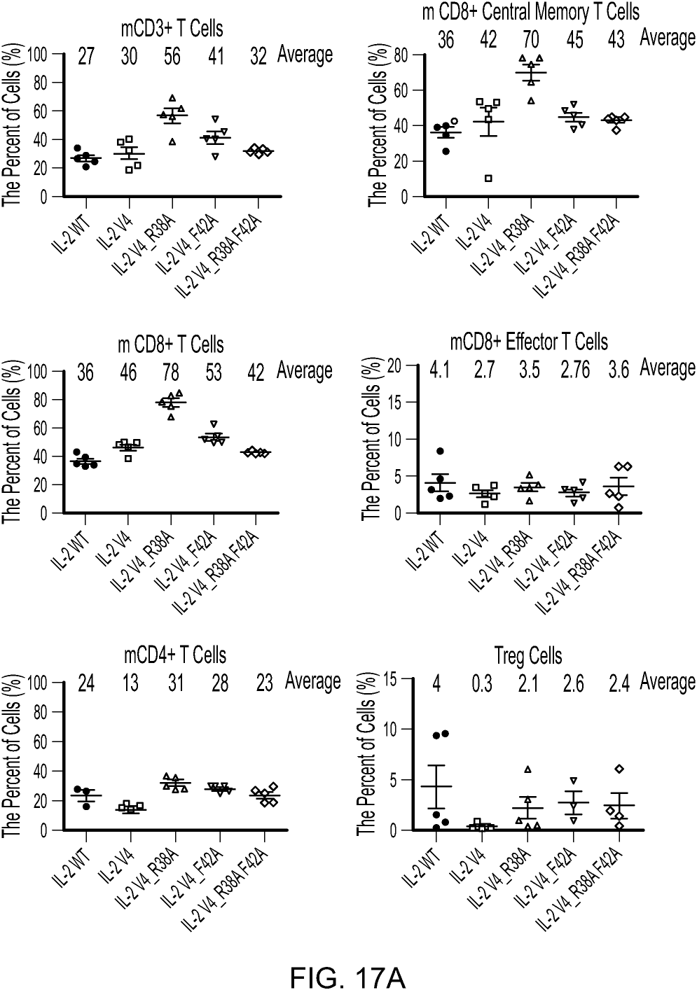

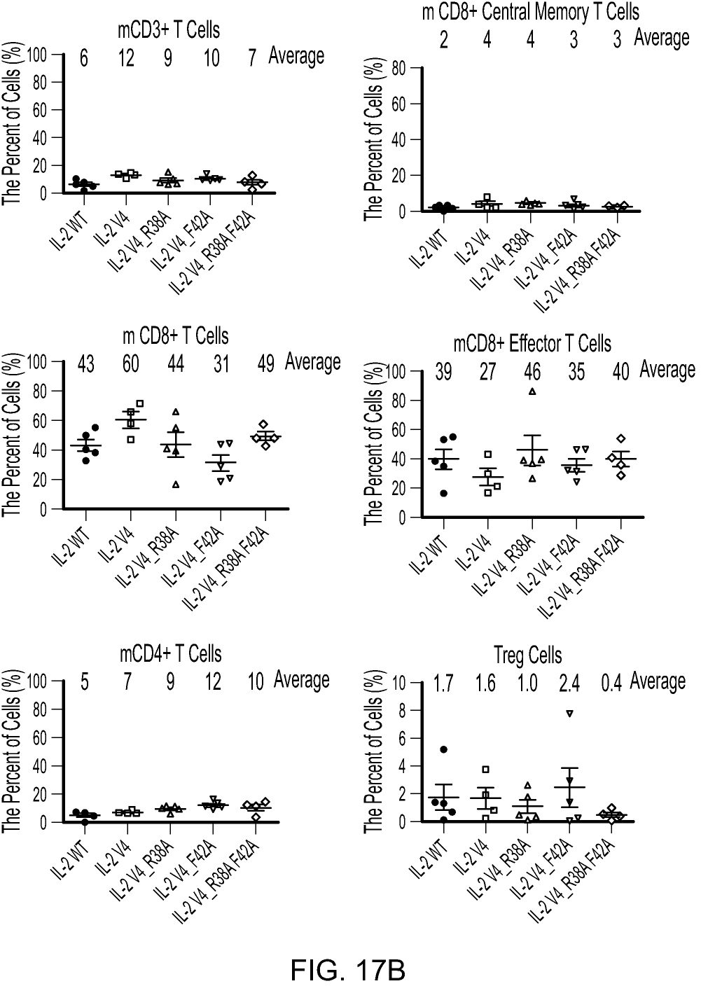

In order to obtain T cells from the blood, Ficoll separation was performed where 300 µL of Ficoll-Paque media was added to a 1.5 mL centrifuge tube. The blood sample from above was carefully layered onto the Ficoll-Paque media solution, then centrifuged at 8,000 rpm for 5 minutes. The opaque white layer containing the lymphocytes was collected and transferred to a 5 mL FACS tube, wherein 3 mL of 1x DPBS buffer was added and then centrifuged at 2,000 rpm for 4 minutes. After discarding the supernatant, the sample was incubated with surface marker fluorescent antibodies for 30 minutes at room temperature. 2 mL FACS buffer was then added to the tube and centrifuged at 2,000 rpm for 4 minutes, where the supernatant was discarded. 1 mL of 1x fix/perm buffer was aliquoted into each tube and incubated in the dark for 30 minutes. 3 mL of 1x perm buffer was then added to each tube and centrifuged at 2,000 rpm for 4 minutes. After the supernatant was discarded, 100 µL of 1x perm buffer and 3 µL of mouse anti-Foxp3 antibodies were added to the tube and reacted for 15 minutes at room temperature. The addition of 1x perm buffer was repeated one more time. The sample was then analyzed using FACS wherein the percent of CD3+ T cells, CD4+ T cells, CD8+ T cells, and T_{reg} cells in blood were measured (FIGS. 7A-7B, 8A-8B, 9A-9C, 10A-10B, 11A-11B, and 17A).

### Tumor Collection and Analysis of Tumor Infiltrating Lymphocytes (TILs)

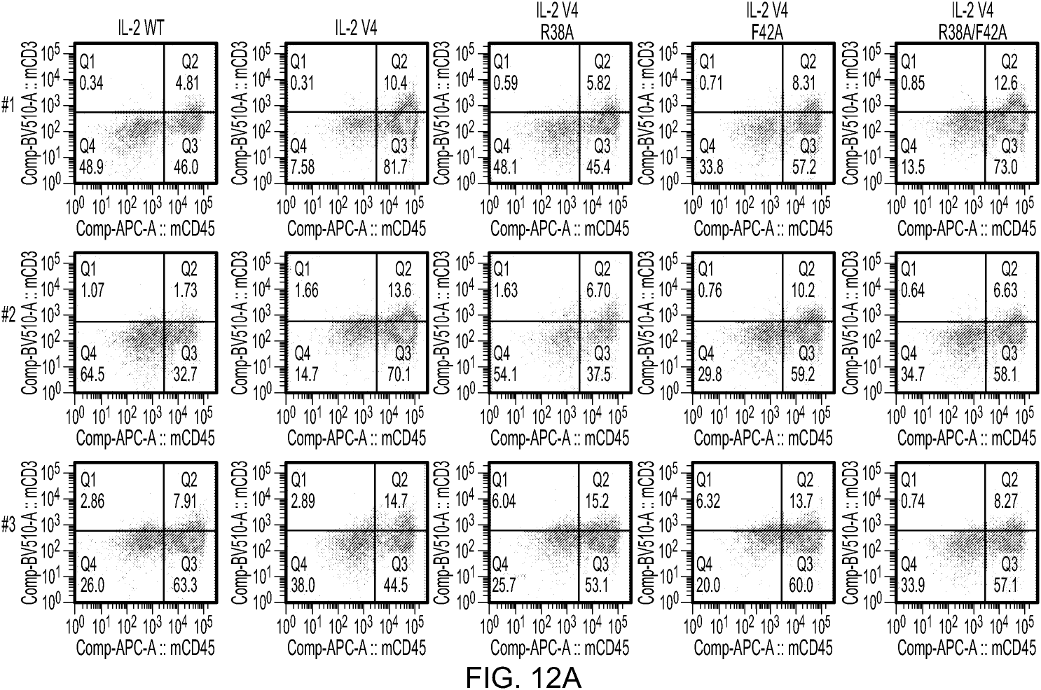

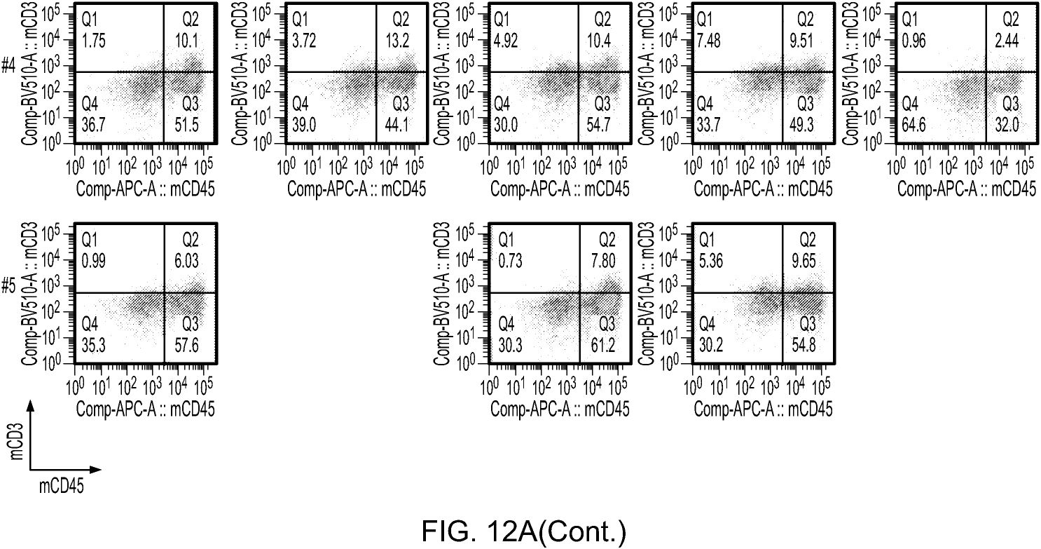

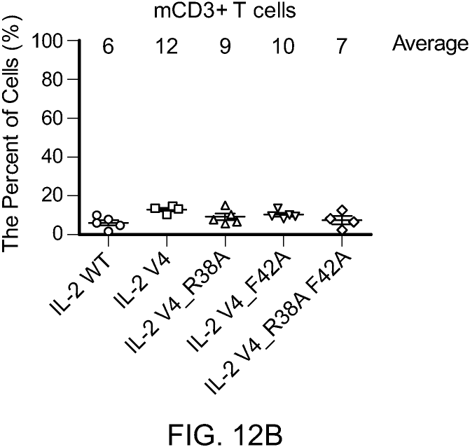

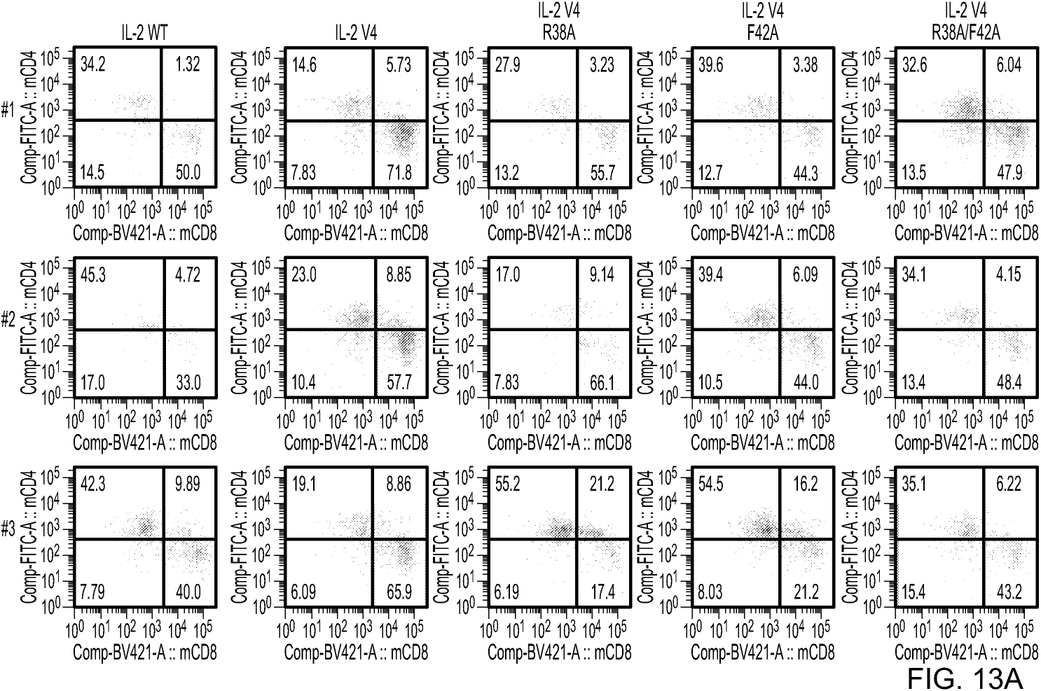

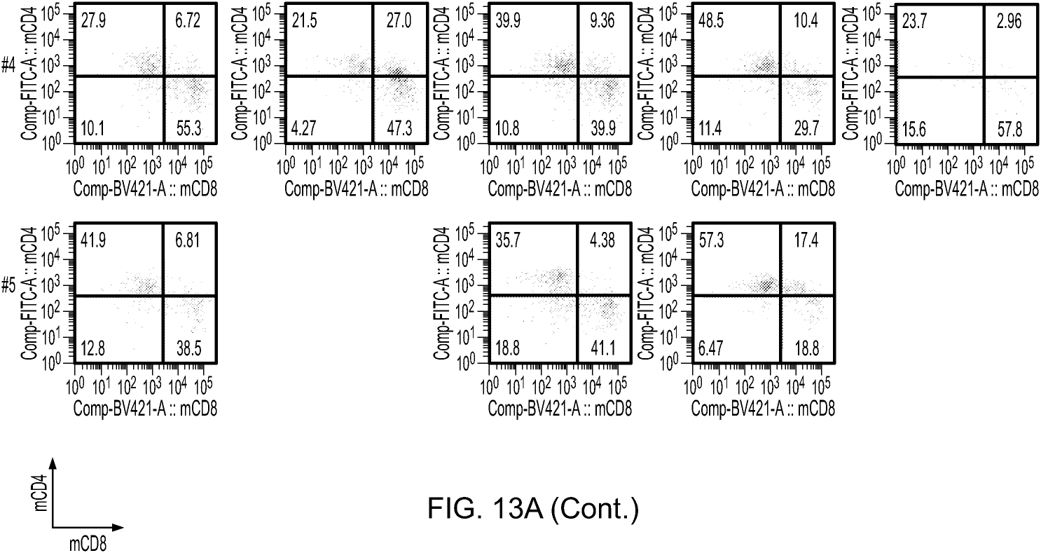

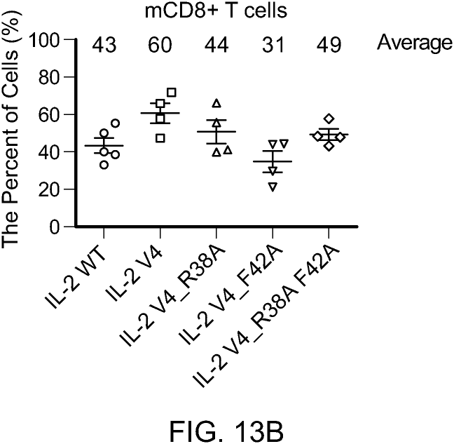

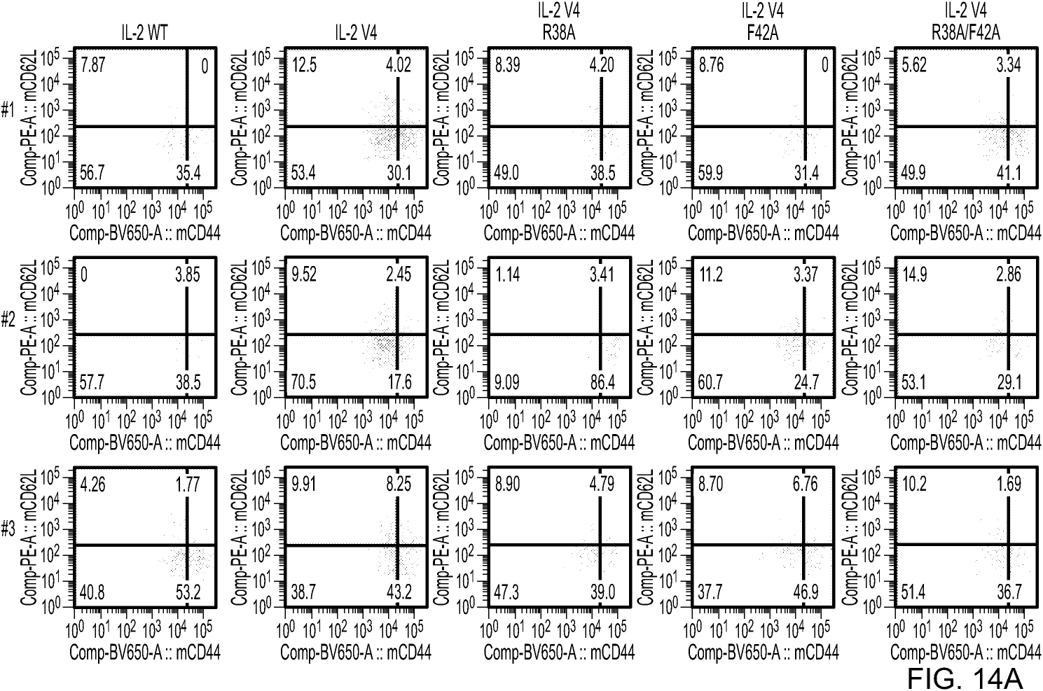

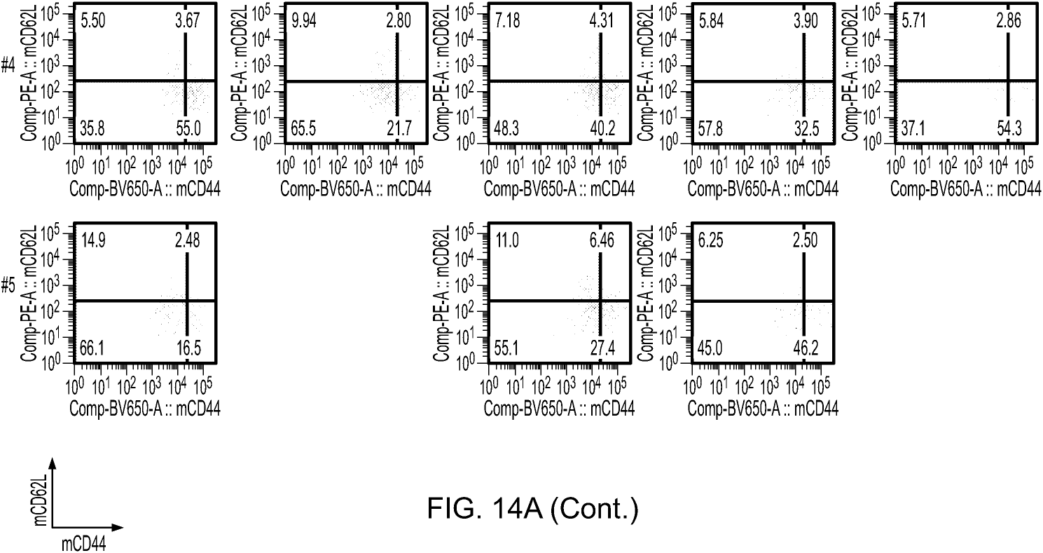

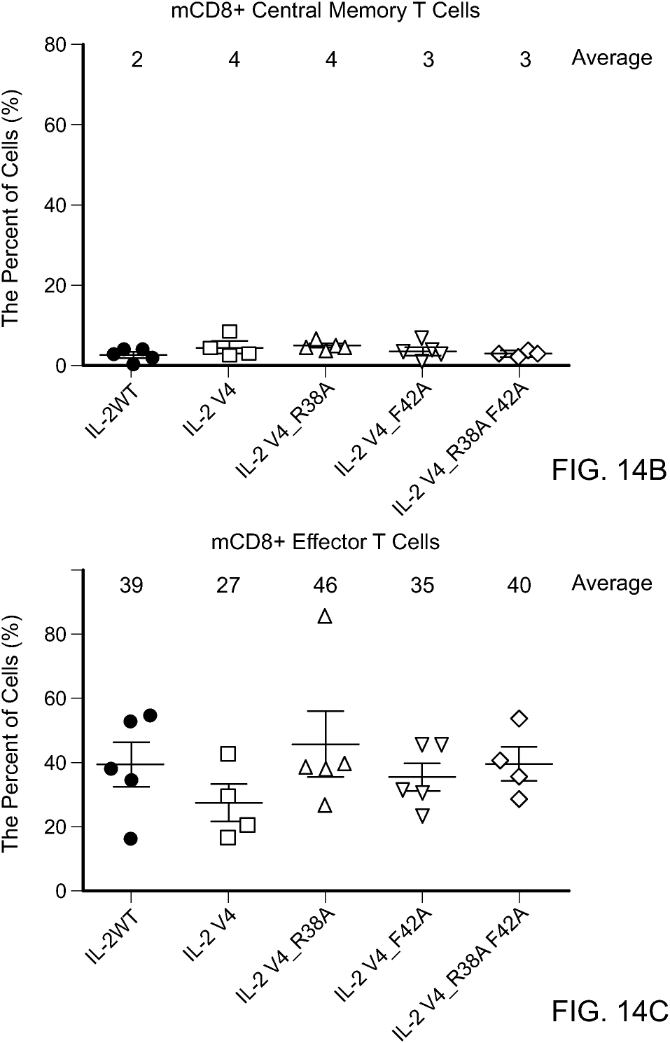

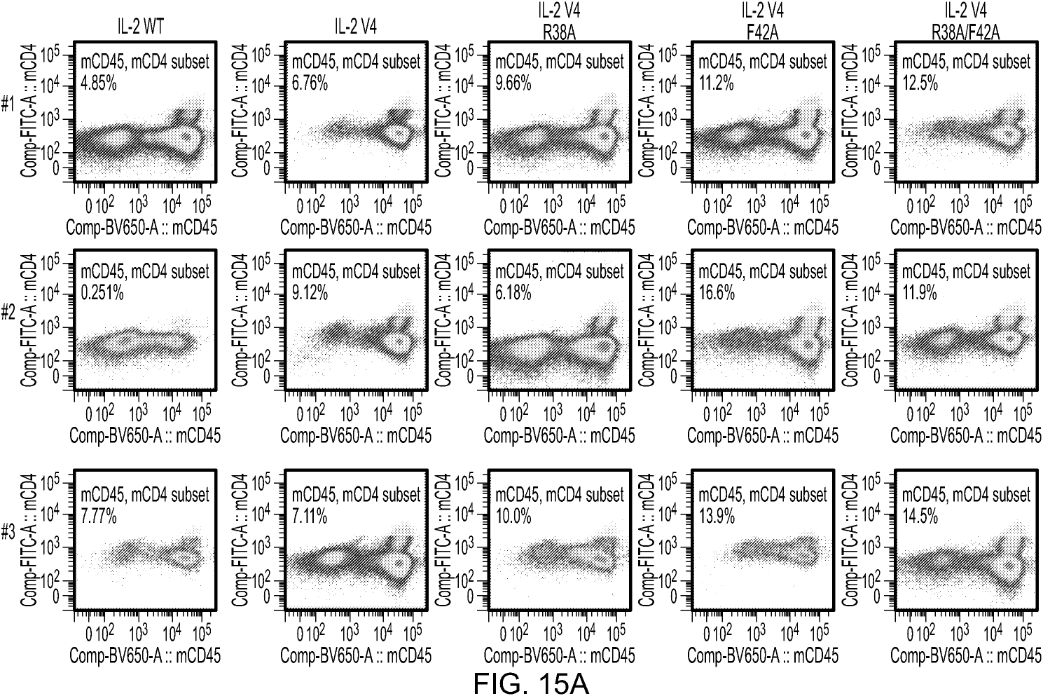

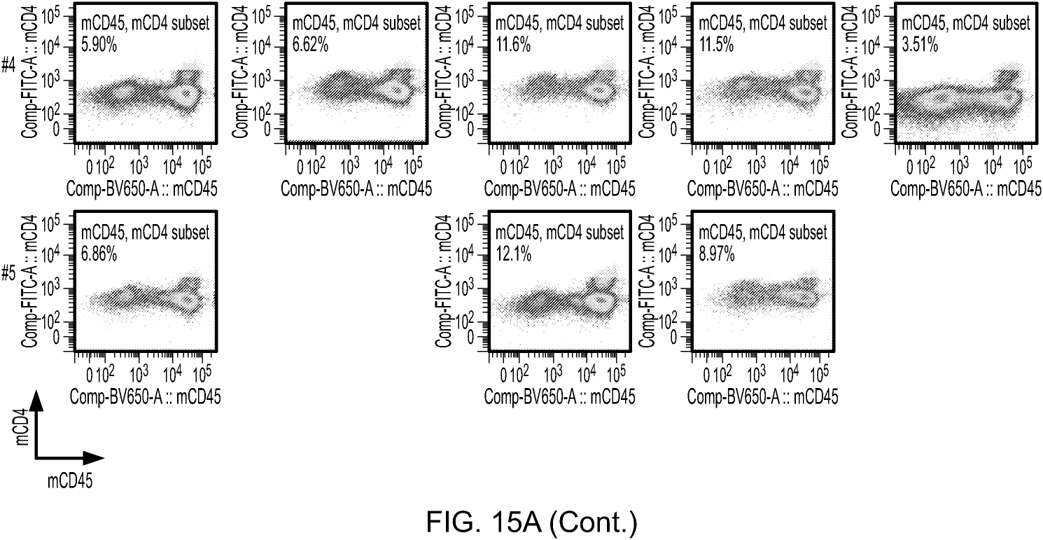

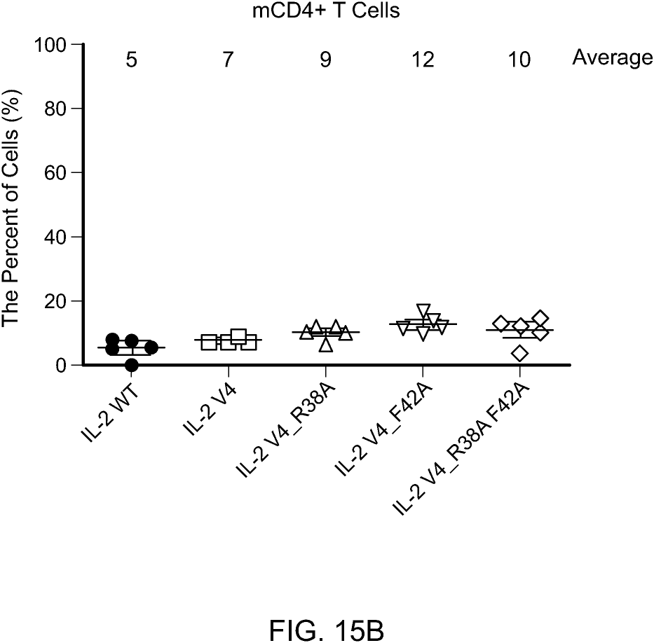

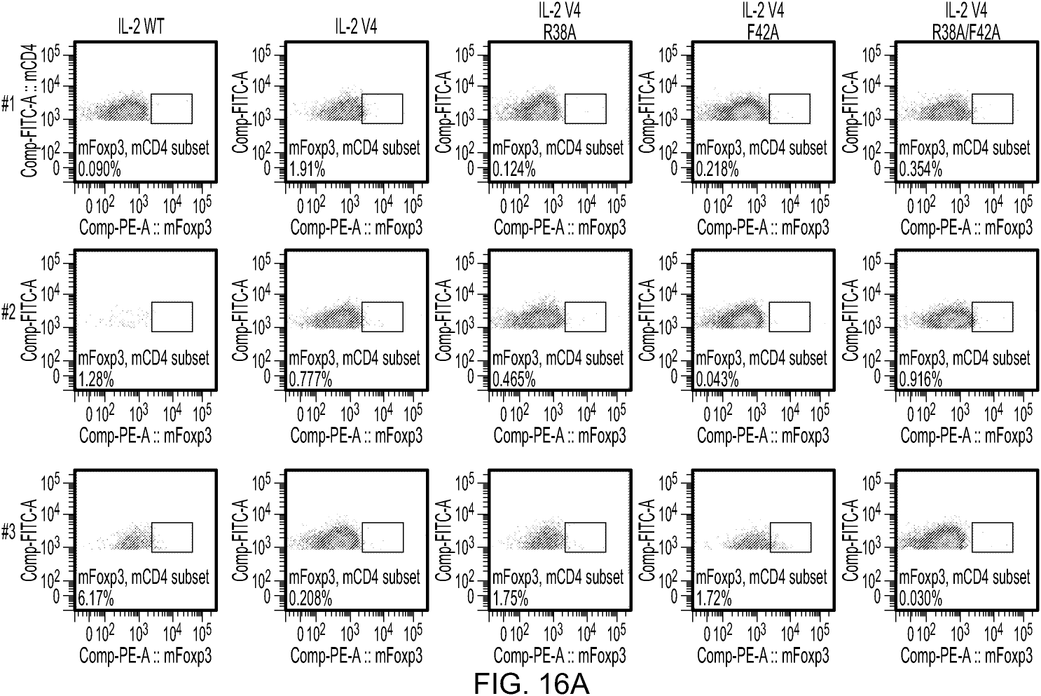

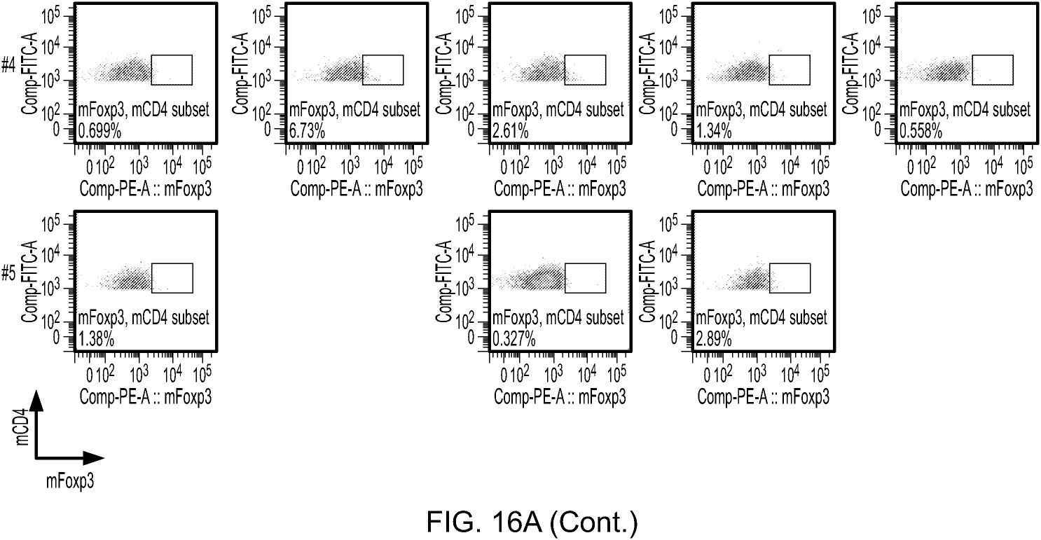

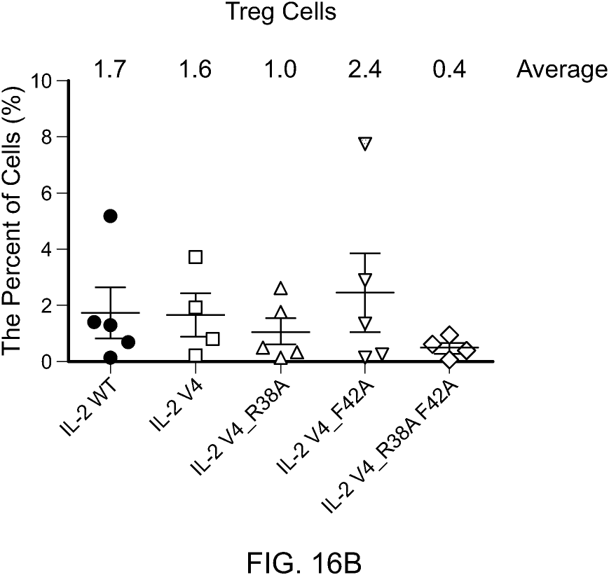

Tissues from the tumors were collected and transferred to a gentleMACS C tube containing RPMI1640 media and the enzyme mix from the tumor dissociation kit. After the GentleMACS program 37C_h_TDK_1 was run for 1 hour, the sample was resuspended in 1x DPBS buffer and the cell suspension was applied to a cell strainer (mesh size 70 µm) placed on a 50 mL tube. The sample was then applied to a cell strainer (mesh size 40 µm) and the above step was repeated until only tumor infiltrating lymphocytes were isolated. After centrifugation at 2,000 rpm for 4 minutes, the supernatant was discarded and 5 mL of ACK lysis buffer was added and incubated for 10 minutes at room temperature. 50 µL of mouse blood was collected and transferred to a 5 mL FACS tube and centrifuged at 2,000 rpm for 10 minutes. The cells were resuspended in 10 mL of RPMI1640 media. The blood sample was then carefully layered onto the Ficoll-Paque media solution, then centrifuged at 2,000 rpm for 30 minutes. The opaque white layer containing the tumor infiltrating lymphocytes (TILs) were collected and transferred to a 50 mL tube, wherein 20 mL of 1x DPBS buffer was added and then centrifuged at 2,000 rpm for 4 minutes. After discarding the supernatant, the sample was incubated with surface marker fluorescent antibodies for 30 minutes at room temperature. 2 mL FACS buffer was then added to the tube and centrifuged at 2,000 rpm for 4 minutes, where the supernatant was discarded and the sample was analyzed using FACS wherein the percent of CD3+ T cells, CD4+ T cells, CD8+ T cells, and T_{reg} cells in tumor infiltrating lymphocytes were measured (FIGS. 12A-12B, 13A-13B, 14A-14C, 15A-15B, 16A-16B, and 17B).

### Toxicity Analysis in Mouse Model

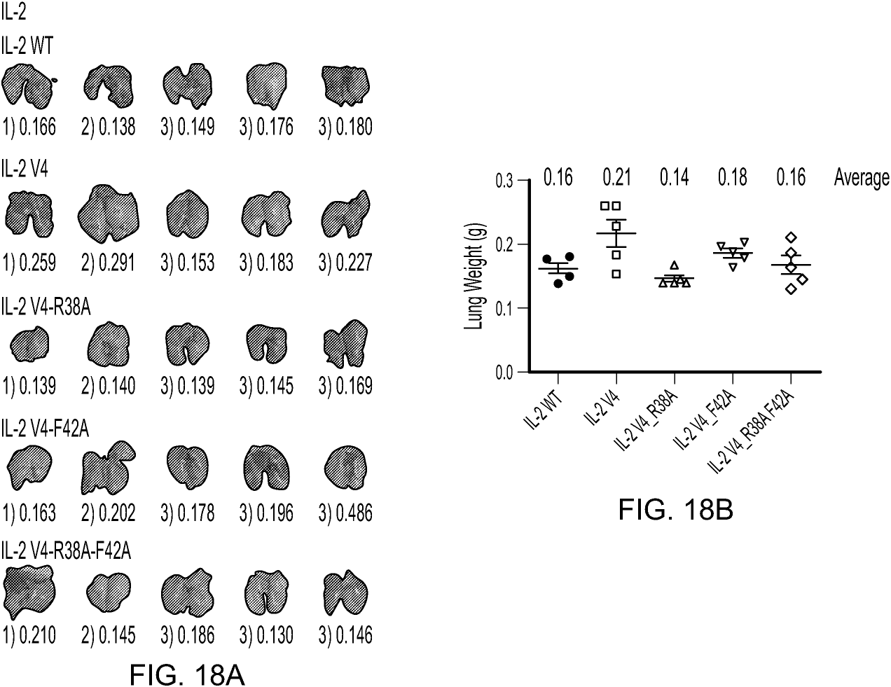

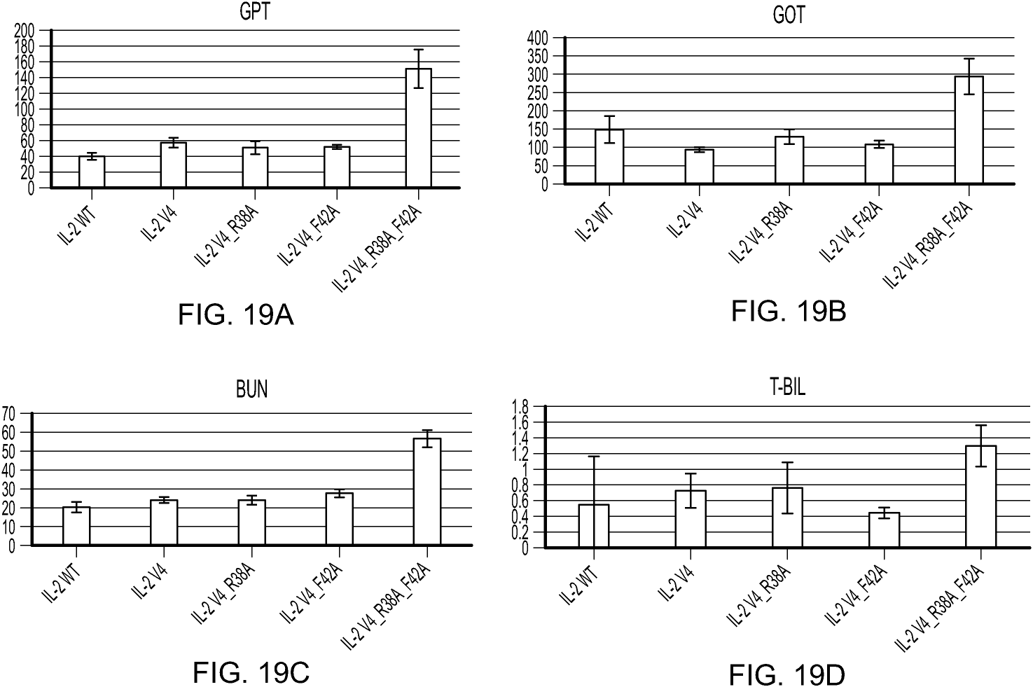

Since development of pulmonary edema has been associated with IL-2 therapy, the toxicity of the IL2 variants was analyzed by collecting lung tissue and blood from the mice. Lung tissue weight was measured and compared for each group to analyze toxicity (FIGS. 18A-18B). Also, blood collected from the mice were centrifuged at 12,000 rpm for 10 minutes at 4° C. to separate the serum. The separated supernatant was transferred to a FUJI DRI-CHEM NX500i tube, wherein alanine aminotransferase (GPT), aspartate aminotransferase (GOT), blood urea nitrogen (BUN), and total bilirubin (T-BIL) levels were measured (FIGS. 19A-19D).

1. An interleukin (IL)-2 protein comprising:
a sequence selected from the group consisting of: SEQ ID NO: 4, SEQ ID NO: 6, SEQ ID NO: 8, SEQ ID NO: 10, SEQ ID NO: 12, SEQ ID NO: 14, SEQ ID NO: 16, and SEQ ID NO: 18.

2. The protein of claim 1, wherein the interleukin-2 protein comprises SEQ ID NO: 4.

3. The protein of claim 1, wherein the interleukin-2 protein comprises SEQ ID NO: 6.

4. The protein of claim 1, wherein the interleukin-2 protein comprises SEQ ID NO: 8.

5. The protein of claim 1, wherein the interleukin-2 protein comprises SEQ ID NO: 10.

6. The protein of claim 1, wherein the interleukin-2 protein comprises SEQ ID NO: 12.

7. The protein of claim 1, wherein the interleukin-2 protein comprises SEQ ID NO: 14.

8. The protein of claim 1, wherein the interleukin-2 protein comprises SEQ ID NO: 16.

9. The protein of claim 1, wherein the interleukin-2 protein comprises SEQ ID NO: 18.

10. The protein of claim 1, further comprising an immunoglobulin Fe region.

11. The protein of claim 10, wherein the immunoglobulin Fe region comprises SEQ ID NO: 20.

12. The protein of claim 11, wherein the immunoglobulin Fe region is linked to the interleukin-2 protein by a peptide bond.

13. The protein of claim 12, wherein the immunoglobulin Fe region is linked to the interleukin-2 protein by a peptide linker sequence.

14. The protein of claim 13, wherein the interleukin-2 protein is linked to the carboxy-terminus of the immunoglobulin Fe region.

15. A pharmaceutical composition comprising the interleukin-2 protein of claim 1 and a pharmaceutically acceptable carrier.

16. A nucleic acid comprising:
a sequence encoding an IL-2 protein, wherein the sequence is selected from the group consisting of: SEQ ID NO: 3, SEQ ID NO: 5, SEQ ID NO: 7, SEQ ID NO: 9, SEQ ID NO: 11, SEQ ID NO: 13, SEQ ID NO: 15,and SEQ ID NO: 17.

17. The nucleic acid of claim 16, further comprising a sequence encoding an immunoglobulin Fe region.

18. The nucleic acid of claim 17, wherein the sequence encoding the immunoglobulin Fe region comprises SEQ ID NO: 19.

19. The nucleic acid of claim 16, wherein the sequence encoding the IL-2 protein comprises SEQ ID NO: 3.

20. The nucleic acid of claim 16, wherein the sequence encoding the IL-2 protein comprises SEQ ID NO: 5.

21. The nucleic acid of claim 16, wherein the sequence encoding the IL-2 protein comprises SEQ ID NO: 7.

22. The nucleic acid of claim 16, wherein the sequence encoding the IL-2 protein comprises SEQ ID NO: 9.

23. The nucleic acid of claim 16, wherein the sequence encoding the IL-2 protein comprises SEQ ID NO: 11.

24. The nucleic acid of claim 16, wherein the sequence encoding the IL-2 protein comprises SEQ ID NO: 13.

25. The nucleic acid of claim 16, wherein the sequence encoding the IL-2 protein comprises SEQ ID NO: 15.

26. The nucleic acid of claim 16, wherein the sequence encoding the IL-2 protein comprises SEQ ID NO: 17.

27. A vector comprising the nucleic acid of claim 16.

28. The vector of claim 27, further comprising a promoter operationally linked to the nucleic acid.

29. The vector of claim 28, wherein the promoter is a constitutive promoter.

30. The vector of claim 28, wherein the promoter is an inducible promoter.

31. The vector of claim 27, wherein the vector is a viral vector.

32. The vector of claim 31, wherein the viral vector is a lentiviral vector.

33. A pharmaceutical composition comprising the nucleic acid of claim 16.

34. A cell comprising the nucleic acid of claim 16.

35. A pharmaceutical composition comprising the cell of claim 34 and a pharmaceutically acceptable carrier.

36. A method of producing an IL-2 protein, comprising:
(a) culturing the cell of claim 34 in a culture medium under conditions sufficient to express the IL-2 protein; and
(b) recovering the IL-2 protein from the cell and/or the culture medium.

37. A method of treating a cancer in a subject, the method comprising administering to the subject a therapeutically effective amount of the pharmaceutical composition of claim 15.

38. The method of claim 37, wherein the cancer is carcinoma, lymphoma, blastoma, sarcoma, leukemia, squamous cell carcinoma, small cell lung cancer, non-small cell lung cancer, lung adenocarcinoma, squamous cell carcinoma of the lung, peritoneal cancer, hepatocellular carcinoma, gastric cancer, pancreatic cancer, glioma, cervical cancer, ovarian cancer, liver cancer, bladder cancer, breast cancer, colon cancer, colorectal cancer, endometrial or uterine carcinoma, salivary carcinoma, kidney cancer, prostate cancer, vulvar cancer, thyroid cancer, liver carcinoma, other lymphoproliferative disorders, or head and neck cancer.

39. The method of claim 37, wherein the cancer is a solid tumor.

40. The method of claim 37, wherein the cancer is colorectal cancer.

41. The method of claim 37, wherein the subject has previously been administered one or more additional anticancer therapies selected from the group consisting of: ionizing radiation, a chemotherapeutic agent, a therapeutic antibody, and a checkpoint inhibitor.

42. The method of claim 37, wherein the subject has been identified or diagnosed as having the cancer.

43. A method of increasing memory CD8+ T cells in a subject, the method comprising administering to the subject a therapeutically effective amount of the pharmaceutical composition of claim 15.

44. A method of increasing CD8+ T cells in a subject, the method comprising administering to the subject a therapeutically effective amount of the pharmaceutical composition of claim 15.

45. A method of decreasing Treg cells in a solid tumor in a subject, the method comprising administering to the subject a therapeutically effective amount of the pharmaceutical composition of claim 15.

46. A method of decreasing a rate of growth of a solid tumor in a subject, the method comprising administering to the subject a therapeutically effective amount of the pharmaceutical composition of claim 15.

47. A method of decreasing volume of a solid tumor in a subject, the method comprising administering to the subject a therapeutically effective amount of the pharmaceutical composition of claim 15.
